(19) United States
(12) Patent Application Publication
(10) Pub. No.: US 2026/0078514 A1
(43) Pub. Date: Mar. 19, 2026

(54) METHODS AND APPARATUS FOR CONTROLLING HYDROGENATION REACTIONS VIA CHARGED PARTICLE STIMULATION OF A HYDROGENATION CATALYST

(71) Applicant: Brillouin Energy Corp., Emeryville, CA (US)

(72) Inventor: Robert E. Godes, Berkeley, CA (US)

(73) Assignee: Brillouin Energy Corp., Emeryville, CA (US)

(21) Appl. No.: 19/402,672

(22) Filed: Nov. 26, 2025

Related U.S. Application Data
(63) Continuation of application No. PCT/US2024/032316, filed on Jun. 3, 2024.
(60) Provisional application No. 63/505,528, filed on Jun. 1, 2023.
(60) Provisional application No. 63/652,279, filed on May 28, 2024.

(51) Int. Cl. C25B 15/025; C02F 1/461; C02F 101/30; C10G 45/72; C10L 1/04; C25B 3/03; C25B 3/07; C25B 3/09; C25B 3/11; C25B 11/02; C25B 11/042; C25B 11/052; C25B 11/067; C25B 15/027; C25C 7/02; C25C 7/06

(52) U.S. Cl. CPC C25B 15/025; C02F 1/46104; C10G 45/72; C10L 1/04; C25B 3/03; C25B 3/07; C25B 3/09; C25B 3/11; C25B 11/02; C25B 11/042; C25B 11/052; C25B 11/067; C25B 15/027; C25C 7/02; C25C 7/06; C02F 2101/30; C02F 2201/4614; C02F 2209/02; C02F 2209/03; C10G 2300/1011; C10L 2290/38; C10L 2290/58

(57) ABSTRACT

Hydrogenation reactions are catalyzed by driving charged particles (e.g., an electric current or particle beam) into and/or through a catalytic material so as to deliver energy for hydrogenation of one or more compounds chemisorbed by the catalytic material. The energy provided by the charged particles may be adjusted (e.g., based on a measured temperature and/or pressure associated with the reaction) to maintain a desired reaction temperature and/or prevent overheating of the reaction. In one example, hydrogen loading of the catalytic material (e.g., via electrolysis) enhances reaction rates. A wide variety of organic and inorganic reactants are contemplated for applications in food, energy production and storage (e.g., fossil-fuels, bio-fuels, petrochemicals, fuel cells), pharmaceuticals and other chemicals, as well as environmental applications (e.g., wastewater treatment, emissions reduction, carbon capture and sequestration).

### CROSS-REFERENCE TO RELATED APPLICATIONS

This application is a bypass continuation of International Application No. PCT/US2024/032316, filed on Jun. 3, 2024, titled “Methods and Apparatus for Controlling Hydrogenation Reactions via Charged Particle Stimulation of a Hydrogenation Catalyst,” which claims a priority benefit, under 35 U.S.C. §119(e), to U.S. Application No. 63/505,528, filed on Jun. 1, 2023, titled “Electrically Driven Hydrogenation Catalyst,” and U.S. Application No. 63/652,279, filed May 28, 2024, titled “Heating Systems and Methods,” each of which is incorporated by reference in its entirety.

### BACKGROUND

Catalyzed reactions typically involve a catalyst that facilitates a reaction between two or more substances to reform at least one of the substances. Some catalyzed reactions may require heat to initiate the reaction and some catalyzed reactions may produce excess heat. One class of catalyzed reactions are hydrogenation reactions that involve molecular hydrogen (H₂) and other compounds, such as organic molecules, to be hydrogenated or saturated. Reactions of this kind generally require the presence of a catalytic material, which commonly take the form of metal particles such as nickel, platinum, palladium, or the like.

The lattice of the catalytic material, under appropriate conditions, dissociates the H₂ gas and holds individual hydrogen atoms at lattice sites, forming intermetallic hydrides and thereby facilitating reactions between hydrogen and the other compounds that can be chemisorbed onto the surface of the catalytic material.

Morphology of the catalytic material can affect reaction rates of hydrogenation reactions. Typically, particles of the catalytic material are used in fluidized bed reactors, where the particles are disposed in a bed (sometimes also referred to as a “compact bed” or “packed bed”) within the reactor through which flows a substance to be hydrogenated. The particles can include a range of sizes, including microscale sizes or even smaller. Hydrogen gas can be bubbled through the bed and the entire mix heated and/or placed under pressure to initiate the hydrogenation reaction. The use of particles can increase the effective surface area of the lattice and can also increase thermal energy (e.g., increased atomic vibration) in the lattice, which can enhance reaction rates.

A drawback of fluidized bed reactors is that they need careful maintenance due to decay of the particles of catalytic material. During use, the particles can deteriorate and form a fine dust that collects in the reactor necessitating routine cleaning of the reactor. If left unclean, the reactor can develop an unsafe, and even potentially explosive, condition. The deterioration of the particles can also render the bed inefficient or inactive after a period of time, such that the bed of particles must be routinely replaced. Further, the fluidized bed reactors must be monitored closely during operation because the reaction can be difficult to control. Thermal run-away is not uncommon in such fluidized bed reactors and must be stopped when detected by shutting down the reactor. The randomness of loose particle activity and non-uniform surface area may affect controllability of the hydrogenation reaction.

### SUMMARY

Disclosed methods and implementations relate generally to catalyzing reactions and electrically enhancing and controlling the catalytic reactions by conducting electrical current along a length of the catalytic material to increase an energy of the lattice of the catalytic material and thereby activate the catalytic reactions. The catalytic reactions can include a wide diversity of hydrogenation reactions. In some implementations, the catalytic material forms part of a transmission line.

One example of catalyzed reactions are hydrogenation reactions. Such hydrogenation reactions can be widely used in several industrial areas including food processing (e.g., to hydrogenate vegetable oils to produce food spreads such as margarine), petrochemical manufacturing and fuel processing (e.g., production of liquid fuels such as diesel), and the production of organic chemical compounds (e.g., polypropylene, vinyl chloride). Hydrogenation processes can be used to convert alkenes, alkynes, aldehydes, imines, and nitriles, into corresponding saturated compounds, such as alcohols and amines. Hydrogenation processes can also be used in the coal processing industry to produce a synthetic liquid hydrocarbon fuel from coal using the Bergius process. Such hydrogenation reactions can be implemented with the systems and methods described in this application.

The inventor has recognized and appreciated that hydrogenation reactions and other catalytic reactions can be improved with a reactor that provides the catalytic material in a uniform, non-particulate morphology. The hydrogenation process can be excited, terminated, and managed in a more controlled manner to avoid thermal run-away with the application of heat and/or electrical current applied to the catalytic material. In some cases, the electrical current can be used to turn on, turn off, and control an amount of the hydrogenation reaction or other catalytic reaction. Because of the catalytic material's non-particulate morphology, dust accumulation and catalyst replacement can occur much less frequently than for the particulate, fluidized bed reactors. Additionally, catalyst replacement can be easily performed when needed.

Certain implementations of the present invention relate to dissolving (or loading) atoms or molecules from a reactive fluid (e.g., hydrogen gas) into a lattice structure (which can comprise a transition metal) for the purpose of catalyzing reactions. The reaction can be, for example, a hydrogenation reaction involving the reactive fluid and a substance to be hydrogenated. The lattice structure can be a self-supporting shape (e.g., in the form of a wire, slab, sheet, strip, tube, screen, mesh, etc.) of solid or sintered material, or can be a catalytic material deposited on such a support structure. In some implementations, the catalytic material can be provided in a powdered or sintered material that is contained in a sitting bed, fluidized bed, or packed bed format such that electrical current can be applied to the catalytic material.

The catalytic material and its lattice can be stimulated to increase its energy, for example, by applying an electrical current to the catalytic material. One way to excite the catalyst is to drive an electrical current through the catalytic material (e.g., along a length of the exposed catalytic material) using a current or voltage source that is electrically coupled to the catalytic material in an electrical circuit. The driven current can be in the form of electrical pulses in some cases. Another way to apply current to the catalytic material is to direct a beam or flux of charged particles (e.g., electrons or ions) having kinetic energy of at least a few eV onto and/or through the catalytic material, thereby providing activation energy to drive the reaction.

Disclosed methods and implementations also relate to catalyzing reactions involving hydrogen to produce heat and electrically enhancing and controlling the catalyzed reactions. The inventor has further recognized and appreciated that the disclosed apparatus can be used to generate heat that can be transferred from the reactor with a heat-transfer fluid for other applications. Such heating systems utilize electrically-driven catalytic tubes to produce heat. The heating systems can, for example, efficiently heat water for commercial, industrial, and residential heating applications (such as radiant floor heating, baseboard hot-water heating, radiator hot-water or steam heating, heated swimming pools, saunas, hot tubs, etc.), though the generated heat can be used for other applications where a heat is needed.

Some implementations relate to an apparatus for controlling a hydrogenation reaction, the apparatus comprising: a reaction chamber to hold molecular hydrogen and at least one compound; a hydrogenation catalyst, disposed in the reaction chamber, to chemisorb at least the at least one compound and catalyze the hydrogenation reaction, the hydrogenation catalyst comprising a transition metal lattice; a supply, electrically coupled to the hydrogenation catalyst, to drive an electric current of charged particles through the transition metal lattice that significantly increases vibrations of atoms in the transition metal lattice so as to deliver an energy to the hydrogenation catalyst and thereby facilitate the hydrogenation reaction via propagation of the energy through the transition metal lattice; at least one sensor to measure at least one of a temperature or a pressure in the reaction chamber associated with the hydrogenation reaction; and a control system, communicatively coupled to the supply, to control the supply so as to adjust the electric current, based at least in part on the at least one of the temperature or the pressure measured by the at least one sensor, to control the energy delivered to and propagating through the transition metal lattice so as to prevent overheating of the hydrogenation reaction.

Some implementations relate to a method for catalyzing a hydrogenation reaction via a hydrogenation catalyst comprising a transition metal lattice. The method can comprise acts of: A) driving an electric current of charged particles through the transition metal lattice so as to significantly increase vibrations of atoms in the transition metal lattice and thereby propagate an energy through the transition metal lattice to facilitate the hydrogenation reaction between molecular hydrogen and at least one compound chemisorbed by the transition metal lattice; B) measuring at least one of a temperature or a pressure associated with the hydrogenation reaction; and C) adjusting the electric current in A) to control the energy propagating through the transition metal lattice, based at least in part on the at least one of the temperature or the pressure measured in B), so as to prevent overheating of the hydrogenation reaction.

Some implementations relate to a method comprising: A) driving charged particles into or through catalytic material of a hydrogenation catalyst so as to deliver an energy to the catalytic material and thereby activate and control a hydrogenation reaction between molecular hydrogen and at least one compound chemisorbed by the catalytic material.

Some implementations relate to an apparatus for controlling a hydrogenation reaction, the apparatus comprising: a reaction chamber to hold molecular hydrogen and at least one compound; a catalytic material, disposed in the reaction chamber, to chemisorb at least the at least one compound and catalyze the hydrogenation reaction; and means for driving charged particles into or through the catalytic material so as to deliver energy to the catalytic material and thereby control the hydrogenation reaction between molecular hydrogen and the at least one compound.

Some implementations relate to systems to provide a catalytic reaction. An example system can comprise: a reactor vessel enclosing a reaction chamber in which the catalytic reaction occurs; at least one input port in the reactor vessel to introduce one or more reactants into the reaction chamber; and a catalyst located in the reaction chamber and arranged such that the catalyst is exposed to the one or more reactants when introduced into the reaction chamber. The catalyst can comprise a layer of catalytic material disposed on a solid support. The layer of catalytic material can be electrically conductive, and the layer of catalytic material can be arranged to conduct electrical current along at least a portion of a length of the layer of catalytic material to increase an energy of the catalytic material and thereby stimulate the catalytic reaction.

Some implementations relate to methods of performing hydrogenation reactions. An example method can include acts of: introducing one or more reactants into a reaction chamber that includes a catalyst disposed in the reaction chamber, the catalyst comprising a catalytic material to catalyze a hydrogenation reaction of the one or more reactants; and driving an electrical current along at least a portion of the length of the catalytic material to increase an energy of the catalytic material and thereby cause the hydrogenation reaction of the one or more reactants to occur.

Some implementations relate to reactor vessels for a system. Such reactor vessels can produce heat from a catalyzed reaction and couple the heat to a heat-transfer fluid. An example reactor vessel can comprise: an outer shell enclosing a vessel chamber to contain a heat-transfer liquid or gas; a containment tube extending through the vessel chamber and through the outer shell, the containment tube sealed to prevent ingress of the heat-transfer liquid or gas into the containment tube; and a catalytic tube to generate heat. The catalytic tube can be mounted within the containment tube and comprise: an electrically-conductive layer extending along the catalytic tube; an insulating layer disposed on the electrically-conductive layer; and an electrically-conductive, reactive layer disposed on the insulating layer, wherein the electrically-conductive layer, the insulating layer, and the electrically-conductive, reactive layer form a transmission line that extends along a length of the catalytic tube, and wherein the containment tube contacts the heat-transfer liquid or gas when the reactor vessel is in operation to thermally couple heat from the catalytic tube to the heat-transfer liquid or gas.

Some implementations relate to methods of heating a fluidic substance with a catalytic tube mounted inside a containment tube, each of which extends through a vessel chamber of a reactor vessel. Such methods can comprise acts of: flowing a reactant over an electrically-conductive, reactive layer disposed on an outer surface of the catalytic tube, wherein the reactant flows in a space between the outer surface of the catalytic tube and an inner surface of the containment tube; propagating electrical pulses along a transmission line disposed on the catalytic tube, the transmission line formed in part by the electrically-conductive, reactive layer; and coupling heat from the containment tube to a heat-transfer liquid or gas flowing within the vessel chamber.

Some implementations relate to electrical connectors for electrically connecting to a transmission line that extends along a catalytic tube, wherein the catalytic tube comprises an exposed first portion of an electrically-conductive layer disposed on an outer surface of the catalytic tube and an exposed first portion of an electrically-conductive, reactive layer disposed on the outer surface of the catalytic tube, wherein the transmission line comprises a second portion the electrically-conductive layer and a second portion of the electrically-conductive, reactive layer. An example electrical connector can comprise: a first clamping plate having a first conical hole formed through the first clamping plate through which the catalytic tube can pass; a second clamping plate having a second conical hole formed through the second clamping plate through which the catalytic tube can pass; a first collet to place over the exposed first portion of the electrically-conductive layer and locate in the first conical hole; and a second collet to place over the exposed first portion of the electrically-conductive, reactive layer and locate in the second conical hole.

Some implementations relate to methods of establishing electrical connection to a transmission line formed on a catalytic tube. The transmission line can comprise: an electrically-conductive layer disposed on a support of the catalytic tube; an insulating layer disposed on the electrically-conductive layer; and an electrically-conductive reactive layer disposed on the insulating layer, wherein the transmission line extends along a length of the catalytic tube. An example method can comprise acts of: engaging, with a first clamping plate of a clamp, a first collet located over an exposed portion of the electrically-conductive layer to reduce an inner diameter of the first collet and thereby establish a first electrical connection between the first collet and the electrically-conductive layer; and engaging, with a second clamping plate of the clamp, a second collet located over an exposed portion of the electrically-conductive reactive layer to reduce an inner diameter of the second collet and thereby establish a second electrical connection between the second collet and the electrically-conductive, reactive layer.

All combinations of the foregoing concepts and additional concepts discussed in greater detail below (provided such concepts are not mutually inconsistent) are part of the inventive subject matter disclosed herein. In particular, all combinations of subject matter appearing in this disclosure are part of the inventive subject matter disclosed herein. The terminology used herein that also may appear in any disclosure incorporated by reference should be accorded a meaning most consistent with the particular concepts disclosed herein.

### BRIEF DESCRIPTION OF THE DRAWINGS

The skilled artisan will understand that the drawings primarily are for illustrative purposes and are not intended to limit the scope of the inventive subject matter described herein. The drawings are not necessarily to scale; in some instances, various aspects of the inventive subject matter disclosed herein may be shown exaggerated or enlarged in the drawings to facilitate an understanding of different features. In the drawings, like reference characters generally refer to like features (e.g., functionally and/or structurally similar elements).

FIG. 1-1A depicts a simplified schematic diagram of a reactor system according to some implementations.

FIG. 1-1B depicts a simplified schematic diagram of a reactor system according to some implementations.

FIG. 1-2 depicts a simplified perspective view of a catalyst according to some implementations.

FIG. 1-3A and FIG. 1-3B depict simplified perspective views of a cylindrical, or tubular, catalyst according to some implementations.

FIG. 1-4 is a flow diagram including at least some steps of a process for operating a reactor system according to some implementations.

FIG. 2-1A depicts a system that comprises catalytic tubes to heat a liquid and/or gas flowing through the heating system or that can be used for a hydrogenation process.

FIG. 2-1B depicts, in perspective view, an example of a reactor vessel that can be used in the system of FIG. 2-1A.

FIG. 2-2A depicts an example of a catalytic tube that includes a reactive transmission line and that can be used in the system of FIG. 2-1A.

FIG. 2-2B illustrates a cross-section of the catalytic tube of FIG. 2-2A.

FIG. 2-2C depicts, in cross-section, the catalytic tube of FIG. 2-2B installed within a containment tube.

FIG. 2-2D depicts another implementation of a reactive transmission line structure and catalytic tube that can be used in the reactor vessel of FIG. 2-1A.

FIG. 2-2E illustrates a portion of the catalytic tube of FIG. 2-2B in finer detail.

FIG. 2-2F depicts another catalytic structure comprising reactive transmission lines that can be used in the reactor vessel of FIG. 2-1A.

FIG. 2-3 depicts further details of a portion of the system of FIG. 2-1A where the catalytic tube passes through an end of the reactor vessel.

FIG. 2-4A depicts an electrical connector that attaches near an end of the catalytic tube of FIG. 2-2A.

FIG. 2-4B depicts an example of a transmission line and contact that can be formed on a PCB used in the electrical connector of FIG. 2-4A.

FIG. 2-4C illustrates further details of collets used in the electrical connector of FIG. 2-4A to make electrical connections to a transmission line formed on the catalytic tube.

FIG. 2-4D is a cross-sectional, perspective view of a collet used in the electrical connector of FIG. 2-4A.

FIG. 2-4E depicts an end of a catalytic tube that can be inserted into the electrical connector of FIG. 2-4A.

FIG. 2-5 illustrates another implementation of a reactor vessel that can be used in the system of FIG. 2-1A.

FIG. 2-6A depicts circuitry connected to a catalytic tube.

FIG. 2-6B depicts another view of the circuitry of FIG. 2-6A connected to the catalytic tube.

### DETAILED DESCRIPTION

Implementations of the present invention relate to dissolving (or loading) a reactive fluid (e.g., hydrogen gas or other liquid or gas) in a lattice structure (e.g., a transition metal lattice) for the purpose of catalyzing reactions involving the reactive fluid. The reactions can include, but are not limited to, hydrogenation reactions and heat-generating reactions. The lattice structure can be a self-supporting shape (e.g., in the form of a wire, slab, sheet, strip, tube, screen, mesh, etc.) of solid or sintered material, or can be a catalytic material deposited on such a support structure. In some implementations, the catalytic material can be provided in a powdered or sintered material that is contained in a sitting bed, fluidized bed, or packed bed format such that electrical current or energetic beam can be applied to the catalytic material.

The lattice structure can be stimulated with electrical current and/or energetic beam of ions or electrons to increase its energy, e.g., by exciting phonons via increased thermal energy of the lattice. Increased thermal energy can be in the form of increased vibrations of atoms in the lattice structure. The increased energy of the lattice can activate catalytic reactions with the lattice and/or increase the rate of catalyzed reactions by the lattice for the reactants. Unlike electrocatalytic reactions in which electrical current is driven through the reactants (e.g., from electrode to electrode via ions in a solution which may contain reactants), the electrical current can be driven along the catalytic material with sufficient energy to excite the catalytic material significantly increasing vibrations of atoms in the lattice. The increased lattice energy is responsible for turning on and/or increasing catalytic activity of the catalytic material. The electrical current may not flow through a solution containing reactants. In some implementations, the electrical current returns to the current source through an electrode that is not exposed to the reactants.

In some cases, electrical pulses can be applied to the lattice with an electrical circuit. A pulse generator can be used to generate the pulses and electrical transmission lines can be used to convey the pulses from the pulse generator to the lattice. In some implementations, a beam of electrons, ions, or other charged particles having kinetic energy of a few eV or higher can be directed onto and/or through the lattice. Particle beams can be generated using a particle accelerator. Any type of particle accelerator can be used, including electrostatic accelerators, wakefield accelerators, or any other accelerator technology that can produce particles having kinetic energy of at least a few eV. The energy delivered by the particle beam to the lattice can provide activation energy to drive a reaction involving the reactive fluid.

### 1.1 Particle Beam Excitation System

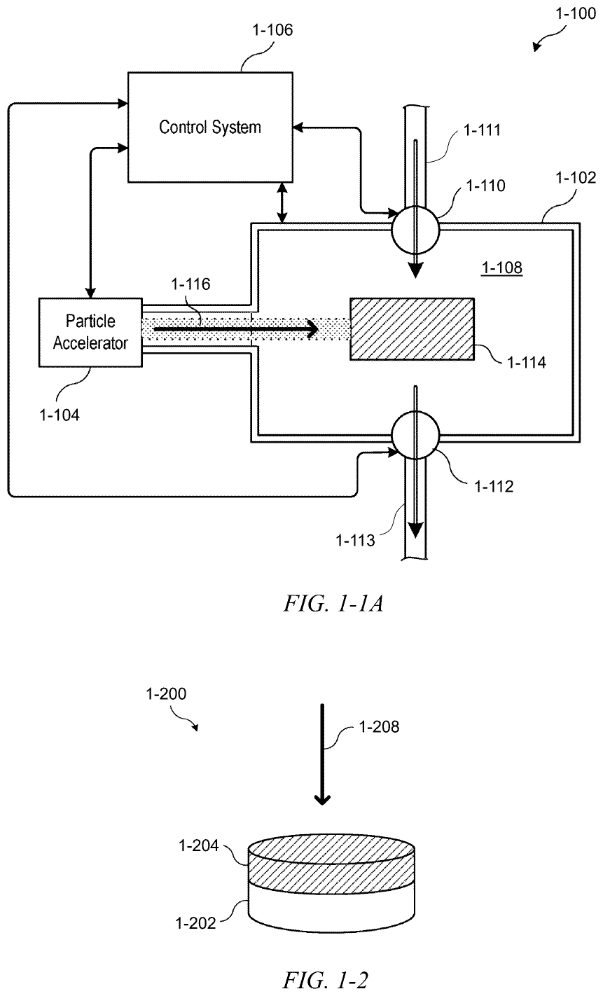

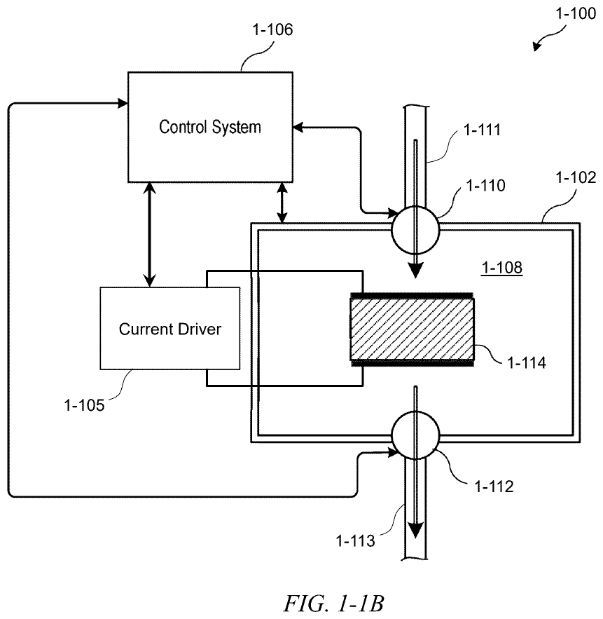

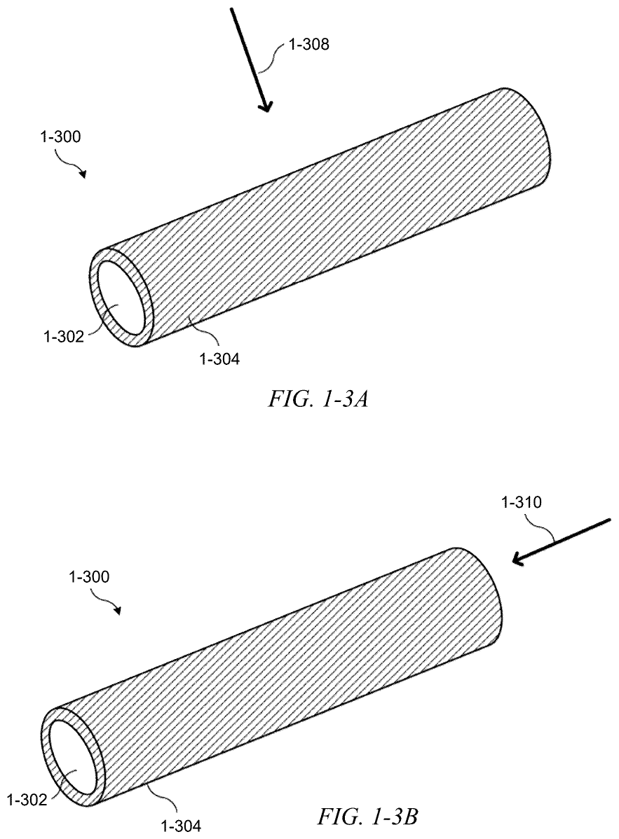

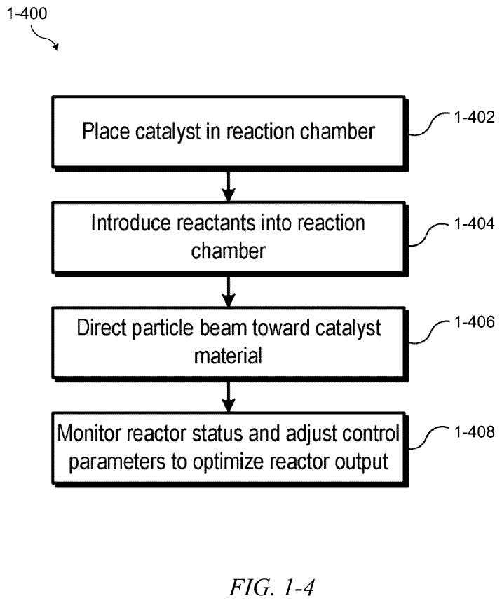

FIG. 1-1A and FIG. 1-1B depict simplified schematic diagrams of a system 1-100 for catalyzing reactions according to some implementations. System 1-100 includes a reactor vessel 1-102, a particle accelerator 1-104 (sometimes referred to more succinctly as an “accelerator”) for the implementation of FIG. 1-1A, a current driver 1-105 for the implementation of FIG. 1-1B, and a control system 1-106. The current driver 1-105 can comprise a voltage source or current source. Reactor vessel 1-102 can provide a reaction chamber 1-108 (e.g., an empty volume defined by the walls of reactor vessel 1-102) in which reactions can occur under controlled temperatures and pressures. The reaction chamber 1-108 can be sealed and contained by the reactor vessel 1-102 such that ambient air surrounding the reactor vessel 1-102 cannot enter into the reaction chamber 1-108 and reaction products cannot escape into the ambient environment. The reactor vessel 1-102 can be formed with metal walls, such as stainless steel for example, and have any desired shape.

Reactor vessel 1-102 can include at least one inlet port 1-110, at least one inlet pipe 1-111, at least one outlet port 1-112, and at least one outlet pipe 1-113, any one or more of which can incorporate valves or other flow control devices. One or more reactants, which can be in a fluid state (e.g., liquid or gas), can be introduced into reaction chamber via inlet port 1-110, and fluids, including reaction products, can be removed via outlet port 1-112.

Reactants can include hydrogen gas or a liquid containing hydrogen and at least one substance to be hydrogenated, for example.

In some implementations, a continuous flow of reactants through reaction chamber 1-108 can be maintained while system 1-100 is active. Alternatively, reactants can be introduced via inlet port 1-110, after which inlet port 1-110 and outlet port 1-112 can be closed while reactions occur. Reaction products (and any residual reactants) can then be pumped out via outlet port 1-112. In some implementations, the same port can be used to introduce reactants and remove reaction products. An external flow control system (not shown in FIG. 1-1A or FIG. 1-1B) can be provided to control the flow of reactants into and out of the reactor vessel 1-102.

A catalyst 1-114 can be placed within reaction chamber 1-108 such that catalytic material of the catalyst 1-114 is exposed to the reactants. Catalyst 1-114 can comprise at least one layer of a reactive catalytic material (also referred to as a “lattice” and “reactive lattice”), such as nickel, palladium, platinum, a transition metal, other catalytic material, or a combination of reactive lattice materials. Catalyst 1-114 can be selected based on the desired reaction to be induced. The catalytic material can reduce the energy required for a reaction to occur without the catalytic material itself reacting with the reactants. Examples of structures and fabrication of catalyst 1-114 are described below. In some implementations, catalyst 1-114 can be a fluidized bed configured to pass an electrical current through the bed.

In some implementations, a charged-particle accelerator 1-104 can be used to impinge a current and particles on the catalyst 1-114, as illustrated in FIG. 1-1A. Accelerator 1-104 can be or comprise a device capable of accelerating charged particles (e.g., electrons or ions) and producing a particle beam 1-116. Particle beam 1-116 can be directed into reaction chamber 1-108 of reactor vessel 1-102 such that the particle beam 1-116 is incident on the catalyst 1-114. In this manner, the particle beam 1-116 can increase thermal energy of (or otherwise impart energy to) the catalytic material of the catalyst 1-114. Accelerator 1-104 can be positioned at any distance from catalyst 1-114. It is noted that shorter beam paths, particularly within reaction chamber 1-108, may reduce unwanted interactions between particle beam 1-116 and reactants or reaction products that may be present in reaction chamber 1-108. Driving charged particles into or through the catalytic material can also be accomplished using pressure or electricity (e.g., electric current). For instance, an electrolytic system, a pressure differential system, or other supply can be used to deliver energy into or thorough the catalytic material.

In some implementations, accelerator 1-104 can be a compact accelerator such as a wakefield accelerator with an output (kinetic) energy in a range from around 100 keV to a few hundred MeV. Other accelerators with other energy ranges can also be used, from a few eV up to the 100 MeV range. In some cases, the accelerator 1-104 can be tunable to different energies within an operating range, and a desired energy of the particle beam 1-116 can be selected for a catalytic reaction. In some implementations, accelerator 1-104 can be operable in a pulsed mode such that the beam is turned off and on, and pulse duration and frequency can be controllable parameters.

Control system 1-106 provides control and monitoring of operation of all other components of system 1-100, including reactor vessel 1-102, inlet port 1-110, outlet port 1-112, and accelerator 1-104. In some implementations, control system 1-106 can be implemented using a microprocessor, microcontroller, other digital logic devices, logic circuitry, analog circuitry, or some combination thereof. Sensors and data communication interfaces can be provided to allow control system 1-106 to monitor the state of various system components and to send control signals to the controllable components. For example, reactor vessel 1-102 can be instrumented with temperature and pressure sensors and with heating and cooling elements (not expressly shown) to control the temperature and pressure of the catalytic reaction process. Control system 1-106 can receive data from the temperature and pressure sensors to monitor conditions inside reaction chamber 1-108 and can send control signals to the heating and cooling elements to adjust the temperature toward a desired temperature. Pressure can be controlled, e.g., by pressure regulating valves, which may be located at inlet port 1-110 and outlet port 1-112. The control system 1-106 can also send control signals to the accelerator 1-104 to control starting and stopping of the particle beam 1-116, including control of pulse duration and repetition rate (or frequency) of a pulsed particle beam. In implementations where the accelerator 1-104 has tunable beam energy, the control system 1-106 can also control the beam energy. In some implementations, the control system 1-106 can include user interface components (e.g., display, keyboard, etc.) to allow a user to specify desired behavior and/or view status information in substantially real time. If desired, the control system 1-106 can be programmed or otherwise configured for autonomous operation, and real-time monitoring by a user is not required.

It should be understood that system 1-100 is illustrative and that variations and modifications are possible. The particular dimensions of the reactor vessel, reaction chamber, and catalyst may be chosen as desired. Operating conditions of temperature, pressure, flow rate of reactants, and particle-beam or electric current stimulation can be determined empirically for a particular reaction using the control system 1-106. Other components can also be provided, such as heat transfer systems using a heat-transfer fluid to remove excess heat from the reactor vessel 1-102. In various implementations, the catalyst 1-114 can be operated as or a solid catalyst with any desired shape.

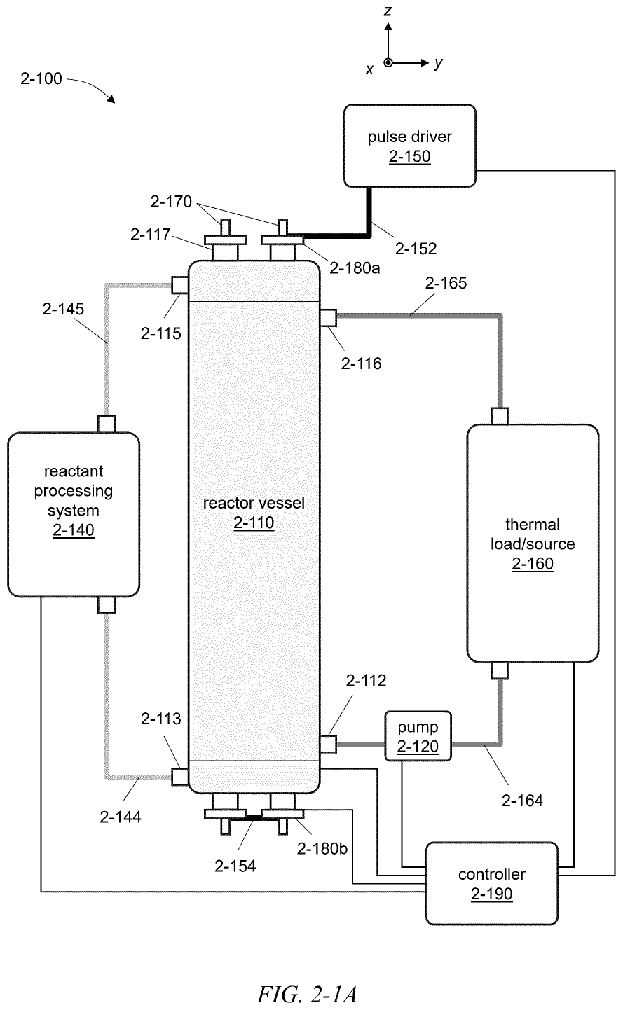

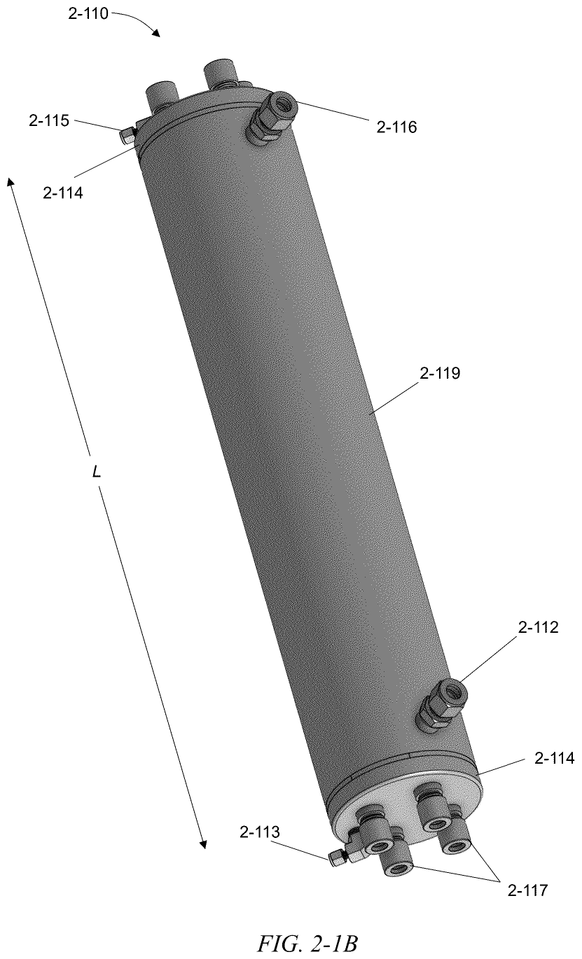

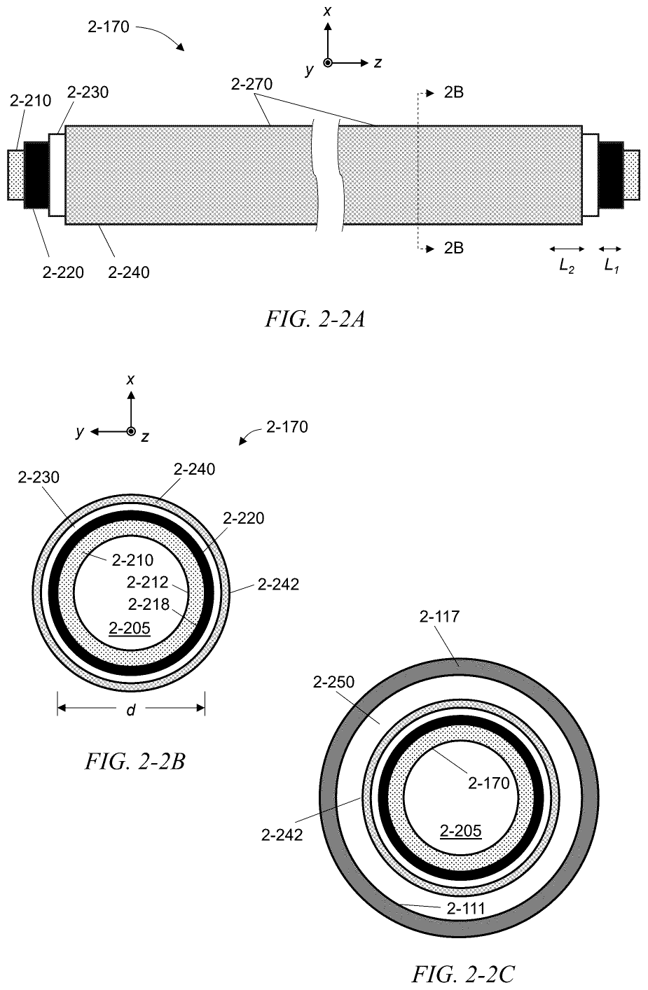

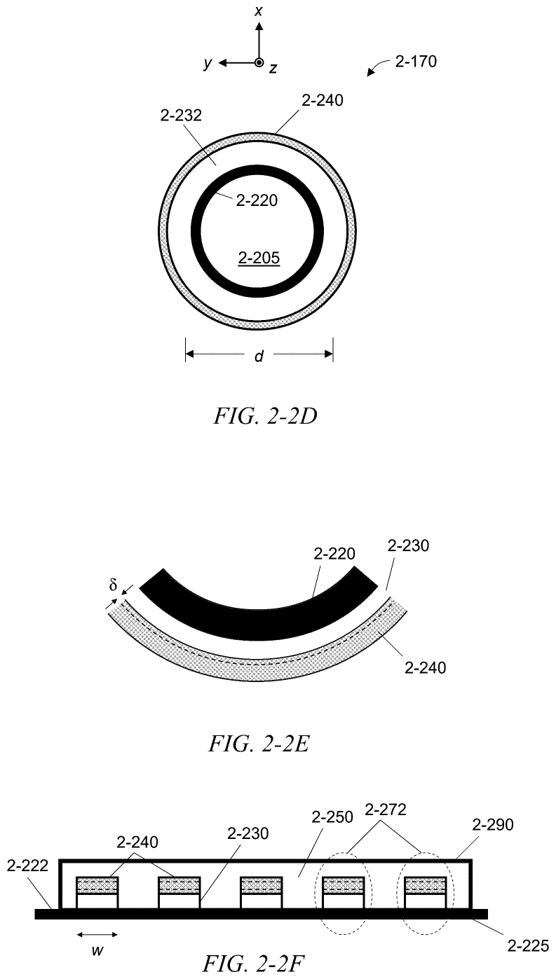

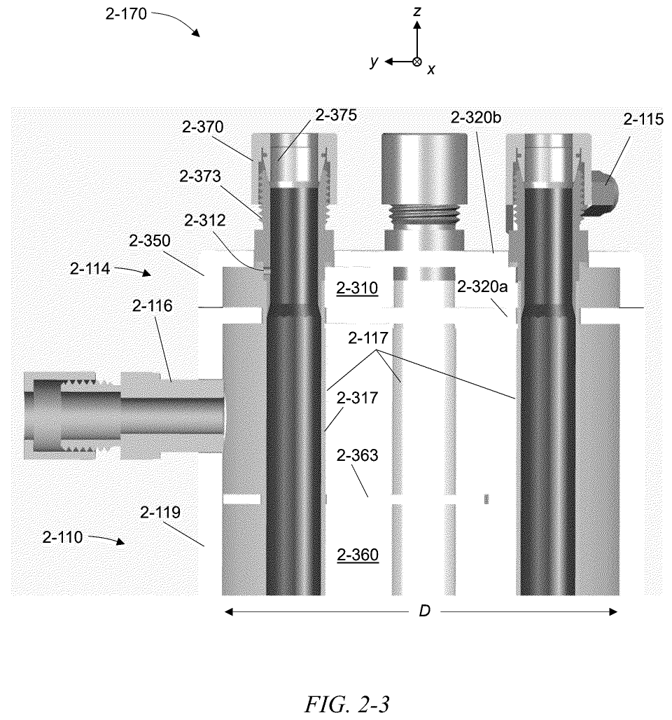

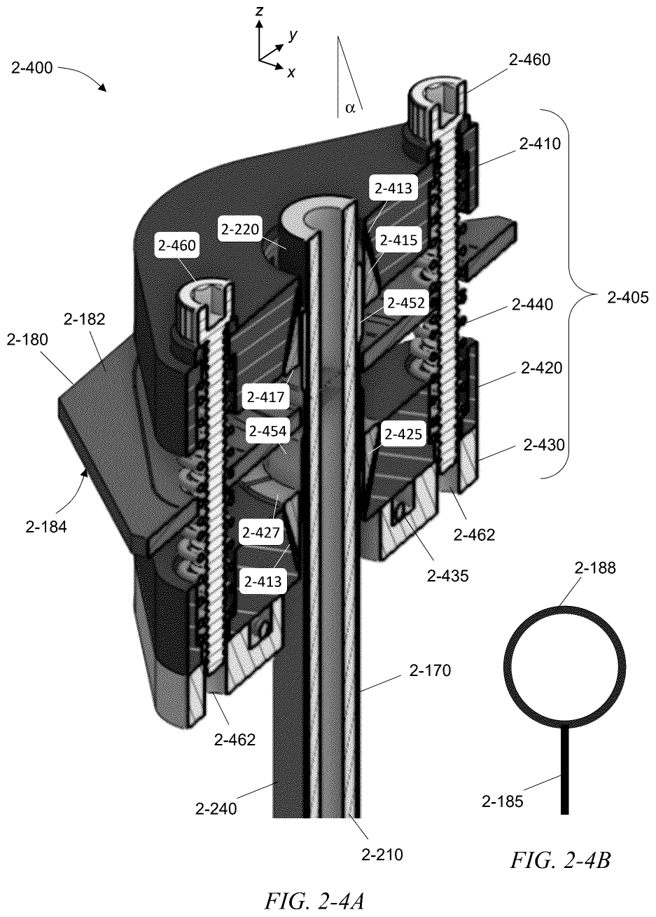

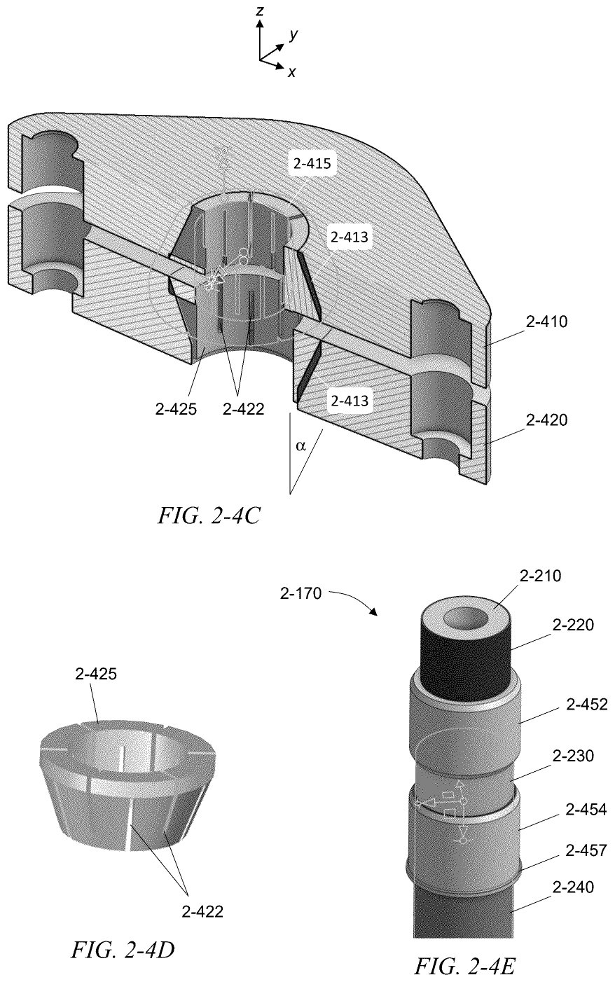

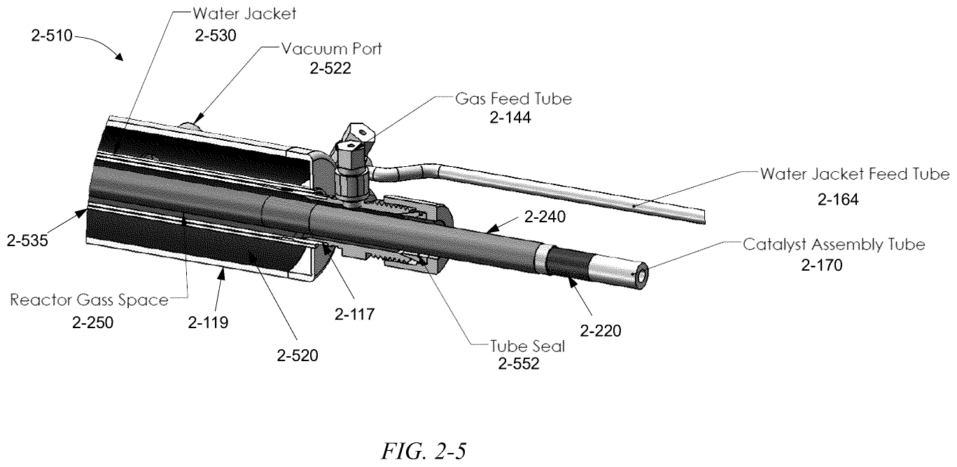

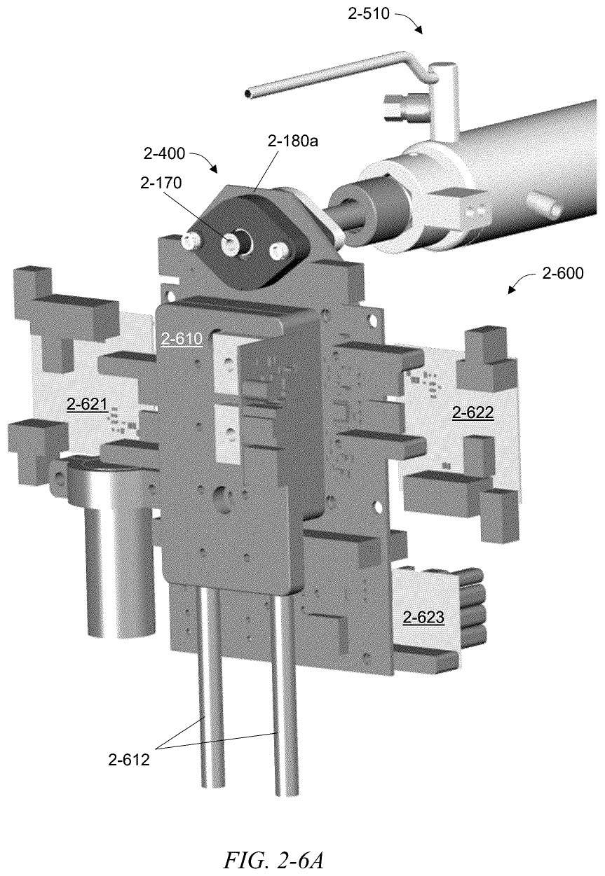

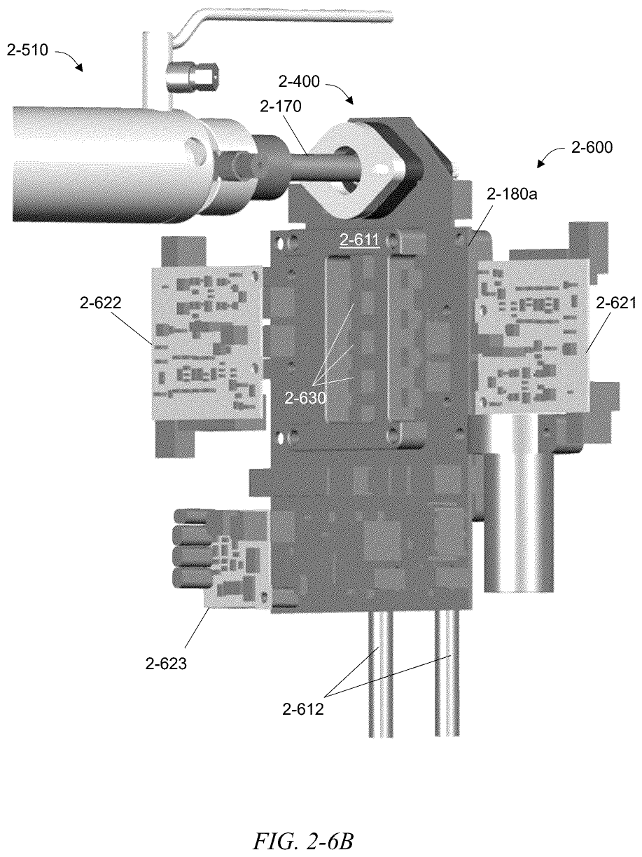

FIG. 1-2 shows a simplified perspective view of a catalyst 1-200 according to some implementations. Catalyst 1-200 can be used as catalyst 1-114 in a reactor system such as system 1-100 described above. Catalyst 1-200 includes a substrate 1-202 (also referred to as a “support”) coated with a catalytic material 1-204 comprising a reactive lattice material.

Substrate 1-202 can be made from various materials depending on the particular application. For instance, substrate 1-202 can be a non-reactive material, such as a ceramic or other dielectric material, which can tolerate the temperature and pressure conditions that are present in reaction chamber 1-108 during operation. In some implementations, substrate 1-202 can be an electrically conductive material that can prevent accumulation of excess charge within the catalytic material 1-204 during exposure to the charged particle beam 1-116, for example.

Catalytic material 1-204 (which may also be referred to as “reactive lattice material,” “lattice material,” or just a “lattice”) can be or include any catalytic lattice material, including a transition metal such as nickel, palladium, or platinum. The lattice can be a solid lattice of bulk lattice material in some implementations. In some cases, the catalytic material 1-204 can be a layer of reactive lattice material deposited on the support or substrate 1-202. The deposited layer can have a uniform morphology to better control and stabilize catalytic reactions. For example, the grain size and surface roughness can each be uniform over the entire exposed surface of the catalyst 1-114 in the reaction chamber 1-108 to within 30% in some cases, to within 20% in some cases, to within 10% in some cases, and even to within 5% in some cases.

In some implementations, the catalytic material 1-204 can be a powdered or sintered lattice, or deposited (e.g., spray-coated or electroplated) material which can absorb reactant atoms (e.g., hydrogen, organic molecules, inorganic molecules, etc.) for a catalytic reaction. To prevent sintering or clumping of catalytic material 1-204 into agglomerates, the powdered or sintered catalytic material 1-204 can be deposited as a layer on the surface of a support structure (e.g., substrate 1-202). The support can comprise a non-reactive matrix of material. In some cases, the powered or sintered catalytic material 1-204 can be deposited on particles composed of a non-reactive or a reactive material, which in turn may be deposited on the surface of the support structure.

In the example structure shown in FIG. 1-2, the catalyst 1-200 comprises a plate with a lattice surface that is roughly planar. While shown as a round plate, catalyst 1-200 can have any shape (e.g., rectangular, square, elongated strips, bars, rods, tubes, sheets, corrugated sheets, etc.). For many hydrogenation reactions, increasing the surface area of the catalytic material 1-204 can increase the amount of catalyzed reactions. Thus, shapes and dimensions of catalyst 1-200 that increase surface area relative to volume within reaction chamber 1-108 of reactor vessel 1-102 are desirable.

In some implementations, the catalyst 1-200 can be oriented within the reactor vessel 1-102 such that the particle beam 1-116 is incident on the planar lattice surface of the catalyst 1-200, as indicated by the arrow 1-208. The particle beam 1-116 can also be incident on other surfaces, such as lateral or side surfaces, depending on the desired lattice response (e.g., intensity and/or directionality of vibrations). In some implementations, the particle beam 1-116 can be scanned across one or more surfaces of the catalyst 1-114. Scanning can be achieved by any relative movement between the catalyst and the particle source or by steering the particle beam (e.g., using variable magnets).

The catalyst 1-200 is not limited to planar surfaces. By way of example, FIG. 1-3A and FIG. 1-3B depict a simplified perspective view of a cylindrical catalyst 1-300 according to some implementations. Like catalyst 1-200, catalyst 1-300 can be used as catalyst 1-114 in a reactor system such as system 1-100 described above. The catalyst 1-300 includes a substrate 1-302 coated with a catalytic material 1-304. The substrate 1-302 can be a rod (as illustrated) or can be a tube. Other shapes of the substrate are also possible.

Like substrate 1-202, substrate 1-302 can be made of a non-reactive material, such as a ceramic or other dielectric material, which can tolerate the temperature and pressure conditions that are present in reaction chamber 1-108 during operation.

Like catalytic material 1-204, catalytic material 1-304 (also referred to as “lattice material” or just a “lattice”) can be or include a metallic lattice material, such as nickel, palladium, platinum, or other transition metal. The lattice can be a solid lattice, or a powdered or sintered lattice, or deposited (e.g., spray-coated or electroplated) material that can absorb hydrogen atoms and/or other reactants. In some implementations, to prevent sintering or clumping of catalytic material 1-304 or reactants, a layer of a porous sintered material can be deposited on the inside surface of the reactor, on a non-reactive matrix, or on particles composed of a non-reactive or another reactive material.

In some implementations, the catalyst 1-300 can be oriented within reactor vessel 1-102 such that particle beam 1-116 is incident on the planar face of catalyst 1-300, as indicated by the arrow 1-308 in FIG. 1-3A. Alternatively, the catalyst 1-300 can be oriented such that the particle beam 1-116 is incident on an end surface, as indicated by the arrow 1-310 in FIG. 1-3B. As with the catalyst 1-200, in some implementations, the particle beam 1-116 can be scanned across one or more surfaces of the catalyst 1-300. In any case, where reactants flow across the catalyst 1-200 or the catalyst 1-300, such flow of reactants can be longitudinal or transverse. The direction of flow of reactants can but need not match the direction of the particle beam 1-116 impinging on the catalyst.

It will be appreciated that catalyst 1-200 and catalyst 1-300 are illustrative, and the catalyst may have any shape as desired. Further, while catalysts 1-200 and 1-300 include lattice material deposited on a supporting substrate, in some cases the lattice material can be self-supporting, and a substrate is not required.

Energy imparted to the catalyst 1-114 via a particle beam 1-116 or electrical current stimulation can propagate throughout the lattice material (e.g., as thermal energy resulting from increased atomic vibrations within the lattice). In some implementations, multiple particle beams can be provided to stimulate different portions of the lattice material or different catalysts disposed inside the same reactor vessel, or to stimulate the lattice material from different directions.

### 1.2 Particle Beam Excitation Methods

FIG. 1-4 depicts steps or acts associated with a process 1-400 of operating a catalytic system, according to some implementations. The process 1-400 can be used to operate a system such as system 1-100 of FIG. 1-1A. Not all steps shown in the drawing need be included in a process of operating the catalytic system. In some implementations of the process, additional steps can be included in the process 1-400.

At block 1-402, a catalyst (e.g., catalyst 1-114) can be placed in a reaction chamber (e.g., reaction chamber 1-108). As described above, the catalyst can include a reactive lattice made of a catalytic material, which can be deposited onto a substrate made of ceramic or other nonreactive material. An appropriate catalytic material (e.g., one comprising nickel, palladium, or platinum) can be used for a desired reaction.

At block 1-404, reactants in a fluid state (e.g., liquid or gas) can be introduced into the reaction chamber. For instance, inlet port 1-110 can be opened to admit reactants into reaction chamber 1-108. The particular reactants can be selected based on the intended reaction and can include hydrogen. (Hydrogen gas may be preferred, given the extreme conditions generally required for liquid hydrogen.) In some implementations, a flow of reactants through the reaction chamber can be established by controlling the inlet and outlet ports. Alternatively, reaction chamber 1-108 can be sealed (e.g., by closing inlet port 1-110 and outlet port 1-112) after introducing the reactants.

At block 1-406, a particle beam can be directed toward the lattice of catalytic material. For instance, accelerator 1-104 can be operated to produce particle beam 1-116. The particle beam can be produced in continuous or pulsed mode as desired. As noted above, the properties of the particle beam can be selected based on the particular reaction being catalyzed and the response of the lattice (e.g., amount of thermal energy generated, particular patterns of vibrations, etc.). Depending on the particular geometry, the particle beam can be incident on a particular point of a lattice surface, scanned across a lattice surface, or directed along a lattice surface.

Process 1-400 can continue as desired to induce reactions in a continuous or ongoing manner. In some implementations, at block 1-408, reactor status can be monitored and control parameters can be dynamically adjusted to optimize output of the reactor system. For example, in some implementations, outlet port 1-112 (or an output line connected to outlet port 1-112) can be instrumented with sensors to detect the concentration of reaction products in the output fluid. Based on the detected concentration, control system 1-106 can operate (autonomously or under human control) to adjust controllable parameters, including the flow rate of reactants through inlet port 1-110; temperature and pressure inside reaction chamber 1-108; and/or properties of particle beam 1-116, including frequency of pulses, pulse duration, and/or beam energy. As another example, if the reactions induced in reaction chamber 1-108 are exothermic (i.e., heat-producing), control system 1-106 can monitor the temperature in reaction chamber 1-108 and/or the temperature of the output fluid and can adjust the controllable parameters to maintain a desired temperature, e.g., to prevent overheating or to maintain an optimal temperature for the reaction to continue.

It will be appreciated that process 1-400 is illustrative and can be modified. For example, instead of using a particle accelerator, a current driver 1-105 (FIG. 1-1B) can be used to drive an electric current through the catalytic material to activate a catalytic reaction. Operations can be performed in a different order from that described, to the extent that logic permits; operations described above may be omitted or combined; operations described sequentially may be performed in parallel; and operations not expressly described may be added. For instance, in some implementations, reaction rates can be enhanced by actively loading hydrogen into the lattice, also sometimes referred to as formation of intermetallic hydrides. Loading of hydrogen can be promoted, for example, by applying a current to the lattice that induces electrolysis. Electrolysis dissociates hydrogen gas into single hydrogen atoms and also drives hydrogen atoms into the lattice. “Loading” currents, electrolysis, and circuitry for generation thereof are all well-understood in the art. Other techniques for inducing dissociation of hydrogen gas and/or driving dissociated hydrogen atoms into the lattice can also be applied. In some implementations, the lattice can be loaded with hydrogen prior to placing the catalyst in the reaction chamber.

Additional processes for catalyzing reactions and related apparatus for catalyzing reactions will now be briefly described.

According to some implementations, a method of catalyzing a reaction can comprise acts of: placing a catalyst comprising a lattice made of a catalytic material in a reaction chamber; introducing one or more reactants to the reaction chamber, the reactants including hydrogen, under conditions that induce loading of hydrogen in the lattice; and directing a particle beam toward the lattice to increase energy (e.g., thermal energy) in the lattice and thereby catalyze a reaction of the reactants.

According to some implementations, another method of catalyzing a reaction can comprise acts of: placing a catalyst comprising a lattice made of a catalytic material in a reaction chamber; introducing one or more reactants to the reaction chamber, the reactants including hydrogen, under conditions that induce loading of hydrogen in the lattice; and driving an electrical current in the lattice to increase energy in the lattice and thereby catalyze a reaction of the reactants.

According to some implementations, a system for catalyzing a reaction can comprise: a reactor vessel including a reaction chamber to hold one or more fluid reactants (such as, but not limited to, hydrogen); a catalyst disposed in the reaction chamber, the catalyst including a lattice made of a catalytic material; and a particle accelerator arranged to direct a particle beam onto the lattice. The particle beam can provide energy to the lattice to catalyze a reaction of the one or more fluid reactants. In some implementations, the system can also include a control system coupled to the particle accelerator and configured to operate the particle accelerator to deliver the particle beam. In some implementations, the reactor vessel can also include one or more ports to allow fluid reactants to enter and exit the chamber. For example, an inlet port can be operable to allow fluid reactants to flow into the reaction chamber, and an outlet port operable to allow fluids including reaction products to exit the reaction chamber. The reactor vessel can operate in a closed configuration in which reactants are introduced through one or more ports, after which the ports are closed while the particle beam is directed onto the lattice or an electrical current is applied to the lattice. After some operating time, the port(s) can be open to extract reaction products. Alternatively, the reactor vessel can operate in a continuous-flow configuration, in which the inlet port and the outlet port are operated by a control system to control a flow rate of the fluid reactants while the particle beam is directed onto the lattice.

According to some implementations, the one or more reactants can be introduced under conditions that induce loading of hydrogen (and/or other reactants) in the lattice of the catalytic material. If desired, the lattice can be loaded with hydrogen (and/or other reactants) prior to placing the catalyst in the reaction chamber.

According to some implementations, the catalyst can include a layer of catalytic material deposited on a substrate. For instance, the catalytic material can include a transition metal, which can be, for example, one or more of palladium, nickel, tungsten, copper, or platinum. Other materials can also be used. The substrate can include a ceramic material, an electrically conductive material (which can prevent accumulation of charge in the lattice), or other material as desired. The lattice can have various shapes. For example, the lattice can be formed to provide a cylindrical surface, a planar surface, or other surface shape.

According to some implementations, a beam of charged particles to excite the catalytic reaction can be provided by a wakefield accelerator, for example, that accelerates charged particles and forms the beam of charged particles. However, other types of accelerators can be used to form a beam of charged particles. The particle beam can be an electron beam, an ion beam, or any other charged particle beam. If desired, the particle beam can be a pulsed beam. The particle beam can have a selected energy (e.g., from at least 1 eV to 1 keV, from at least 1 keV to 1 Mev, or from at least 1 MeV to at least 10 MeV). The particle beam can be incident on the lattice from various directions, including on an end surface of the lattice, or directed along a surface of the lattice.

While the aspects of catalyzing hydrogenation reactions and catalyzing heat-generating reactions are described with reference to certain implementations, those skilled in the art will appreciate that variations and modifications are possible. For instance, the structure of the reactor vessel and control systems can be varied as desired, depending on considerations such as the particular reaction and a desired peak reaction rate. In some implementations, multiple particle beam sources can be provided. For example, where multiple catalysts are placed within the same reactor vessel, multiple beam sources can be arranged to direct beams toward the lattice material of each catalyst. As another example, multiple beam sources can be arranged to direct beams toward the lattice material from different directions to allow further control of thermal energy added to the lattice.

Some implementations described above assume that a particle beam is created by producing charged particles (e.g., electrons or ions) at a location external to the lattice and accelerating or steering the charged particles toward the lattice. Such charged particles may flow through the lattice, similarly to an electrical current. Accordingly, the same catalytic effect as a particle beam can be achieved by applying a voltage across or along the lattice, thereby inducing an electrical current in the lattice (e.g., with the system of FIG. 1-1B). In some cases, the electrical current comprises a series of electrical pulses. In some implementations, the electrical current may be applied as a DC current for a period of time that is not pulsed on and off during its application. In some implementations, the electrical current can be applied as an alternating current (AC) at a selected frequency in a range from 10 Hz to 20 GHz. In some implementations, the electrical current can be applied at an acoustic frequency (or at several acoustic frequencies) to stimulate an acoustic wave in the catalytic material.

Catalytic materials can include any material capable of loading reactants. Examples include magnesium, chromium, iron, cobalt, nickel, molybdenum, palladium, silver, tungsten some ceramics, etc.

The structure and materials used for the catalyst can be chosen as desired, based on the particular reaction to be catalyzed. In some implementations, a single substrate may be patterned with multiple regions of catalytic material that are separated from each other, and a particle beam can be directed onto each such region.

Depending on the particular reaction, the reactor system can be operated in a “flow” mode, in which reactants flow into (and out of) the reaction chamber while the catalyst is being stimulated, or in a “closed” mode, in which reactants are introduced and then the reaction chamber is sealed (e.g., by closing any inlet and/or outlet ports) prior to stimulating the catalyst to perform a reaction cycle. Reaction products can be removed at the end of the reaction cycle (e.g., by opening an outlet port). In some implementations, a single port can serve as both inlet and outlet.

A variety of reactions can be induced using techniques and structures of the kind described herein. Examples include hydrogenation reactions, in which hydrogen is first trapped by the lattice of catalytic material, then bonded to organic molecules or inorganic molecules that are chemisorbed onto the lattice surface, as well as any other reactions for which formation of an intermetallic hydride is a step in the process.

### 2-1. Catalytic Tube System

The inventor has further recognized and appreciated that catalytic reactions can be stimulated in catalytic tubes through which a reactant flows. The reactions can be stimulated by temporally short, wideband electrical pulses. In some cases, the stimulated reactions can efficiently produce heat which can be transferred to a heat-transfer substance that flows in close proximity to and/or in contact with the catalytic tubes. In such cases, the catalytic tubes can be incorporated into a heating system designed to heat the heat-transfer substance that flows through the system. Such a system can be used, for example, to efficiently produce heated water for commercial and residential heating applications (such as domestic hot water, radiant floor heating, baseboard hot-water heating, radiator hot-water or steam heating, heated swimming pools, saunas, hot tubs, etc.), though heating of other liquids or gases for other applications are also possible. The inventor has also recognized and appreciated that such catalytic tube systems can be used for hydrogenation processes which may be used in the food and chemical industries.

FIG. 2-1A depicts an example of a system 2-100 that comprises a reactor vessel 2-110 having catalysts formed as catalytic tubes 2-170 to catalyze a reaction. In some cases, the catalyzed reaction can generate heat that is used to heat at least one heat-transfer substance (a fluidic substance such as a liquid and/or gas) flowing through the reactor vessel 2-110. In some implementations, the catalyzed reaction can be a hydrogenation reaction. The catalytic tubes 2-170 extend through the reactor vessel 2-110 and are located within containment tubes 2-117, which also extend through the reactor vessel 2-110. The illustrated system 2-100 further comprises a pump 2-120, an electronic pulse driver 2-150, a reactant processing system 2-140, and a controller 2-190. In some implementations, the heat-generating reaction can be one as described in U.S. patent application Ser. No. 11/617,632, filed on Dec. 28, 2006, titled “Energy Generation Apparatus and Method,” which application is herein incorporated by reference in its entirety, or in U.S. patent application Ser. No. 14/189,751, filed on Feb. 25, 2014, titled “Control of Low Energy Nuclear Reaction Hydrides, and Autonomously Controlled Heat,” which application is herein incorporated by reference in its entirety.

When implemented as a heating system, the heat-transfer substance output from the reactor vessel 2-110 can be provided to a thermal load 2-160. In some implementations, the thermal load 2-160 can be a hot-water system for a commercial or residential application (e.g., domestic hot water, radiant floor heating, baseboard hot-water heating, radiator hot-water or steam heating, heated swimming pools, saunas, hot tubs, etc.). In such implementations, the reactor vessel 2-110 can heat the water that is distributed by the hot-water system.

When implemented as a hydrogenation system, the heat-transfer substance circulated through the reactor vessel 2-110 can have its temperature set and controlled by a thermal source 2-160. For example, the hydrogenation process may be heated to a temperature to facilitate catalytic reactions on the catalytic tubes 2-170.

The controller 2-190 can be communicatively coupled, through wireless and/or wired links, to at least one other component in the system 2-100 to implement monitoring and/or control functionality for the system 2-100. The controller 2-190 can comprise at least one processing device and may comprise a combination of processing devices. Example processing devices that can be used in the controller 2-190 include, but are not limited to: microprocessor, microcontroller, programmable logic controller (PLC), field-programmable gate array (FPGA), digital signal processor (DSP), application-specific integrated circuit (ASIC), digital logic chips, and transistors. In some cases, the controller 2-190 can further include discrete electronic components (e.g., resistors, capacitors, inductors, etc.), at least one display element (e.g., light indicator, liquid-crystal display (LCD), an LCD or LED monitor, a touchscreen, etc.), and at least one input element (e.g., a touchscreen, keypad or keyboard, button, switch, etc.). In some implementations, the controller 2-190 comprises a tablet computer, laptop computer, smartphone, or other packaged computing device. The controller can be co-located with the system 2-100 (e.g., attached to the reactor vessel 2-110), or can be located remotely from the heating apparatus (e.g., communicatively coupled over a network, such as a local area network or wide area network). In some implementations, portions of the controller 2-190 can be co-located with the system 2-100 and portions of the controller 2-190 can be located remotely from the heating apparatus.

In the illustration of FIG. 2-1A, the controller 2-190 is communicatively coupled to the reactor vessel 2-110, the pump 2-120, the reactant processing system 2-140, the pulse driver 2-150, and the thermal load 2-160. Each of these communicative couplings can include one or more control lines or channels for issuing control signals to a connected device. Additionally, or alternatively, there can be one or more sense lines or channels to communicatively couple the controller 2-190 to one or more sensors and one or more of the connected devices (e.g., reactor vessel 2-110, pump 2-120, reactant processing system 2-140, and pulse driver 2-150) in the system 2-100. The sense lines or channels can be used to receive data from the connected sensor or connected device.

The pump 2-120 can comprise a liquid or gas pump that is operated to circulate a heat-transfer substance through the reactor vessel 2-110 and thermal load or source 2-160. The heat-transfer substance can flow out at least one output heat-transfer port 2-116 from the reactor vessel 2-110 and via at least one output heat-transfer line 2-165 through the thermal load or source 2-160. When implemented as a heating system, cooled heat-transfer substance can return from the thermal load 2-160 via at least one return heat-transfer line 2-164 to at least one input heat-transfer port 2-112 and thus flow back into the reactor vessel 2-110 for reheating and continued circulation. In some cases, one or more output heat-transfer lines 2-165 and one or more return heat-transfer lines 2-164 can connect to one or more of the catalytic tubes 2-170 and to the thermal load or source 2-160 for additional heat transfer, as described further below in connection with the catalytic tubes.

The reactant processing system 2-140 can manage flow of a reactant gas or liquid for the catalytic tubes 2-170. The reactant processing system 2-140 can include at least one reservoir to hold the reactant gas or liquid. The reactant processing system 2-140 can include at least one pump to circulate the reactant gas or liquid through or over the catalytic tubes 2-170 and through apparatus within the reactant processing system 2-140 (such as filters). The reactant processing system 2-140 can include various apparatus to process the reactant gas or liquid (e.g., one or more filters, gas sensors, pressure regulators, etc.). The reactant gas or liquid can be provided to one or more catalytic tubes 2-170 in the reactor vessel 2-110 via at least one reactant input line 2-144 and at least one reactant input port 2-113 and return to the reactant processing system 2-140 via at least one reactant output port 2-115 and at least one reactant return line 2-145.

When implemented as a hydrogenation system, the reactant processing system 2-140 can further manage flow of a substance to be hydrogenated. For example, the reactant processing system 2-140 can circulate a substance to be hydrogenated through the reactor vessel 2-110 and extract a hydrogenated product output from the reactor vessel 2-110. In some implementations, the reactant processing system 2-140 can introduce hydrogen gas or a liquid comprising hydrogen into the substance to be hydrogenated (e.g., by mixing and/or bubbling) prior to entry into the reactor vessel 2-110, such that the substance and hydrogen gas or liquid are mixed and conveyed along a same reactant input line 2-144 to the reactor vessel 2-110. In some implementations, the reactant processing system 2-140 can introduce hydrogen gas or a liquid comprising hydrogen into the substance to be hydrogenated after to entry into the reactor vessel 2-110, such that the substance and hydrogen gas or liquid are separately conveyed to the reactor vessel 2-110 along separate reactant input lines 2-144 and mixed or bubbled within the reactor vessel 2-110.

The electronic pulse driver 2-150 is configured to output electrical excitation pulses to at least one catalytic tube 2-170 in the reactor vessel 2-110. In some implementations, there can be more than one electronic pulse driver 2-150 per reactor vessel 2-110. For example, each catalytic tube can have a dedicated electronic pulse driver 2-150 connected to it. The electronic pulse driver 2-150 is configured to output sequences of temporally short, broad frequency spectrum pulses (also referred to as Q pulses) to excite reactions in the catalytic tubes and thereby cause the generation of heat in the catalytic tubes 2-170. The electronic pulse driver 2-150 can be used to turn on, turn off, and control an amount of heat generation or hydrogenation reaction with the reactor vessel 2-110. Examples of drive circuitry for an electronic pulse driver 2-150 are described in U.S. Pat. No. 8,624,636 titled “Drive Circuit and Method for Semiconductor Devices,” issued Jan. 7, 2014, which patent is herein incorporated by reference in its entirety. In particular, the circuitry shown and described in connection with FIG. 3A, FIG. 3B, FIG. 7A and FIG. 7B of that patent can be used in the pulse driver 2-150 to produce short pulses with short turn-on and turn-off transitions.

In some implementations, heat can be provided to the reactor vessel 2-110 by a heat-transfer fluid and/or electrical heater coupled to the vessel's catalyst. The supplied heat may be nearly enough, but not enough, to initiate a catalytic reaction. Application of the electrical current (from the electronic pulse driver 2-150, for example) may provide enough excitation to the catalytic material of the catalytic tubes 2-170 to initiate the catalytic reaction. Adjusting the applied current (e.g., increasing an average amount, increasing peak values, and/or changing pulse length) may control an amount of the catalytic reaction. Terminating the applied current can terminate the catalytic reaction.

In some implementations, the pulse driver 2-150 can be configured to produce excitation pulses that each rise quickly from an initial value to a peak value, sustain approximately the peak value for a period of time, and fall back to the initial value. Examples of such pulses are square pulses, though other pulse shapes are possible. The excitation pulses can be output from the pulse driver 2-150 at a repetition frequency f with a duty cycle D (ratio of the pulse's on time to period T=1/f). The rise time τᵣ of the excitation pulses can be less than approximately or exactly 50 ns (e.g., between approximately or exactly 1 ns and approximately or exactly 50 ns), though shorter rise times can be used in some implementations. The repetition frequency f can be between approximately or exactly 1 kHz and approximately or exactly 500 kHz and the duty cycle D can be between approximately or exactly 0.5% and 50%. The peak amplitude of the excitation pulses can be between approximately or exactly 20 V and approximately or exactly 1000 V.

In some cases, the pulse driver 2-150 can be configured to produce excitation pulses that each rise to a peak value and fall to an initial value without a sustained duration of the peak value. Examples of such excitation pulses are Gaussian pulses, though other pulse shapes are possible. The temporal full-width-half-maximum value τₒ of the excitation pulses can be less than approximately or exactly 200 ns (e.g., between approximately or exactly 1 ns and approximately or exactly 200 ns), though shorter excitation pulses can be used in some implementations. The repetition frequency f can be between approximately or exactly 1 kHz and approximately or exactly 500 kHz and the duty cycle D can be between approximately or exactly 0.5% and 50%. The peak amplitude of the excitation pulses can be between approximately or exactly 50 V and approximately or exactly 1000 V.

The inventor has recognized and appreciated that the application of pulses to the transmission line of the catalytic tube 2-170 can produce phonons in the lattice of the catalytic material. These phonons shake the lattice and can trigger heat-producing reactions catalyzed by the lattice material. More abrupt rise times and/or shorter pulse durations of the applied electrical pulses comprise a broader band of frequencies that can excite the lattice and increase reactivity of the catalytic tube 2-170.

The electrical pulses from the electronic pulse driver 2-150 can be applied to the catalytic tubes 2-170 via transmission lines 2-152, 2-154, which can be implemented as radio-frequency (RF) coaxial cables. In some cases, the transmission lines 2-152, 2-154 can connect to printed circuit boards (PCB) 2-180a, 2-180b (also referred to as “coupling PCB 2-180a, 2-180b” or more generally as “coupling PCB 2-180”) which in turn electrically couple to the catalytic tubes 2-170. According to some implementations, as illustrated in FIG. 2-1A, the electronic excitation pulses can be applied via a first transmission line 2-152 to an end of a first one of the catalytic tubes 2-170, travel down the first catalytic tube to an opposing end and then be applied via a second transmission line 2-154 to an end of a second one of the catalytic tubes 2-170. In this manner, the electronic excitation pulses can be applied to all of the catalytic tubes 2-170 (which are connected in series) in the reactor vessel 2-110. In some cases, the pulse driver 2-150 can output pulses for exciting reactions in at least two catalytic tubes 2-170 that are connected in series (e.g., from 2 to 8, from 5 to 20, or more than 20). In some cases, the pulse driver 2-150 can provide excitation pulses to more than 8 catalytic tubes 2-170 that are connected in series. In some implementations, the pulse driver 2-150 can provide excitation pulses to multiple catalytic tubes 2-170 that are connected in parallel (e.g., from 2 to 8, from 5 to 20, or more than 20).

FIG. 2-1B illustrates, in perspective view, an example of a reactor vessel 2-110 that can be used in the system of FIG. 2-1A. The reactor vessel is tipped to show an end of the reactor vessel 2-110. There are four containment tubes 2-117 extending from each end of the reactor vessel 2-110 which can house four catalytic tubes 2-170 (not shown in FIG. 2-1B), though a reactor vessel 2-110 can have fewer or more containment tubes 2-117 than shown in the drawing. The reactor vessel comprises an outer shell 2-119 and feed-thru manifolds 2-114 located at each end of the reactor vessel 2-110. To reduce heat loss to the external environment and increase an amount of heat coupled to or from the heat-transfer liquid or gas flowing through the reactor vessel, the reactor vessel 2-110 can be wrapped in insulation and/or heat-reflective material and covered with a thin layer of sheet metal, polymer, or other material (not shown in the drawing) in an arrangement similar to household hot water heaters. The length L of the reactor vessel 2-110 can be from 50 cm to 200 cm, though shorter or longer reactor vessels can be implemented.

### 2-2. Catalytic Tubes

FIG. 2-2A and FIG. 2-2B depict an example of a catalytic tube 2-170 that can be used in the system of FIG. 2-1A. FIG. 2-2B illustrates a cross-section (taken at the dashed line) of the catalytic tube 2-170 of FIG. 2-2A. In this illustrated implementation, the catalytic tube 2-170 comprises a support 2-210 and several layers of material deposited on the support. The support 2-210 can be formed from an electrically conductive or non-conductive material that is able to withstand temperatures of up to 800° C. without permanently deforming or being damaged. The layers deposited on the support 2-210 can include, but are not limited to, an electrically-conductive layer 2-220, an electrically-insulating layer 2-230 disposed on the electrically-conductive layer 2-220, and an electrically-conductive, reactive layer 2-240 disposed on the electrically-insulating layer 2-230. In some implementations, a second electrically-insulating layer can be deposited between the support 2-210 and the electrically-conductive layer 2-220, to increase electrical and/or thermal isolation of the support 2-210 from the outer layers.

The phrase “disposed on” can mean that a second layer physically contacts an underlying first layer (e.g., is physically deposited on and is in intimate contact with the underlying first layer). The phrase “disposed on” also can mean that a second layer is disposed over the underlying first layer with at least one in intervening layer between the first layer and second layer.

The support 2-210 can be a cylindrical tube or have another shape (e.g., a square tube, rectangular tube, polygonal tube, or elliptical tube). The support 2-210 can extend at least the length L of the reactor vessel and can further extend beyond each end of the reactor vessel 2-110 when installed in a containment tube 2-117 of the reactor vessel 2-110. The support 2-210 can have an interior wall 2-212 surrounding a hollow core 2-205. In other implementations, the support 2-210 may not have a hollow core and instead be solid or porous at its interior region. When the support 2-210 has a porous or hollow core 2-205, a heat-transfer substance (e.g., a liquid or gas) can be circulated through the hollow core 2-205 and through a thermal load 2-160 with additional fluidic connections between the catalytic tubes 2-170, the thermal load 2-160. The heat-transfer substance can be circulated with the same pump 2-120 or an additional pump. Alternatively, an electrical heating element can be placed inside the hollow core 2-205 to heat the catalytic tube 2-170 and facilitate a hydrogenation reaction, for example.

According to some implementations, the support 2-210 is formed from a metal (e.g., stainless steel). Other materials that can be used to make the support 2-210 include, but are not limited to, invar, Zerodur®, a ceramic, alumina, fused silica or other glass, zirconia, and sapphire. In some implementations, the outer diameter d or maximum transverse dimension of the support 2-210 or catalytic tube 2-170 can be between approximately or exactly 2.5 mm and approximately or exactly 12 mm, though larger diameters may be used in some cases.

Having a small diameter can be beneficial for some catalytic reactions that are stimulated with a high peak current density (about 2 kA/mm²) in the reactive layer 2-240 and for which the pulse driver 2-150 operates with voltages below 1000 volts. For some applications, the peak current density driven in the reactive layer 2-240 by the pulse driver 2-150 is between approximately or exactly 1.5×10³ A/mm² and 3×10³ A/mm². For a pulse driver 2-150 operating at higher voltages and/or supplying higher currents, the support 2-210 can have larger diameters or transverse dimensions (e.g., up to 25 mm or even larger). The length of the support 2-210 (along the z direction in the drawings) can be between approximately or exactly 40 cm and approximately or exactly 220 cm. With smaller diameters, more than four containment tubes 2-117 (and catalytic tubes 2-170 installed therein) can be mounted in the reactor vessel 2-110 to increase heat output. For example, up to 100 containment tubes 2-117 and catalytic tubes 2-170 could be assembled into a reactor vessel 2-110.

For hydrogenation processes and other catalytic reactions, the reactions may be activated and controlled with lower current densities in the reactive layer 2-240 (e.g., from 0.1 kA/mm² to 1 kA/mm² or even lower current densities). For some cases, the current density through the catalytic material can be from 1 A/mm² to 10 A/mm² or from 10 A/mm² to 100 A/mm². In such systems, the outer diameter d or maximum transverse dimension of the support 2-210 or catalytic tube 2-170 can be larger than 12 mm (e.g., from 12 mm to 50 mm or even larger).

FIG. 2-2C depicts a cross-section of the catalytic tube 2-170 of FIG. 2-2B installed within the containment tube 2-117. An annular reaction chamber 2-250 (similar to the reaction chamber 1-108 of FIG. 1-1A and FIG. 1-1B) exists between the outer surface 2-242 of the catalytic tube 2-170 and in inner surface 2-111 of the containment tube 2-117. A reactant gas or liquid can flow along the catalytic tube 2-170 in a sheath over the outer surface 2-242 and contact the outer surface of the catalytic tube. In a hydrogenation process, the substance to be hydrogenated can also flow along the catalytic tube 2-170 in a sheath over the outer surface 2-242 of the catalytic tube 2-170. As such, the flowing reactant(s) can contact the electrically-conductive, reactive layer 2-240 of the catalytic tube 2-170.

For some materials, such as stainless steel, the support 2-210 and catalytic tube 2-170 of FIG. 2-2A may undesirably sag when operating at temperatures up to 800° C. (e.g., when the catalytic tube is mounted such that its length is oriented horizontally in a heater installation). To prevent sag, the annular reaction chamber 2-250 between the reactive layer 2-240 of the catalytic tube 2-170 and the inner wall of the containment tube 2-117 (when the catalytic tube 2-170 is installed inside the containment tube 2-117) can be packed with a porous, thermally-conductive fill material, such as alumina. The containment tube 2-117 can then provide additional support to the catalytic tube 2-170. The porous material can still allow flow of the reactant(s) within the annular reaction chamber 2-250.

In other implementations, the structure of the catalytic tube can be different than that shown. In one example, the structure of the catalytic tube is reversed from that shown in FIG. 2-2B, such that the support tube is located on the outside of the catalytic tube 2-170 and the layers are deposited on the interior of the outer support such that the electrically-conductive, reactive layer is in inner most layer adjacent to the hollow core 2-205. In this implementation, the reactant(s) can flow through the hollow core 2-205. The containment tube 2-117 can be omitted from the reactor vessel 2-110 in such an implementation so that the heat-transfer substance contacts the outer support tube.

In another example, illustrated in FIG. 2-2D, an insulating tube (such as a fused quartz tube) 2-232 can be used to provide support for the catalytic tube 2-170, provide electrical isolation between the conductive layers, and form the reactive transmission line with the electrically-conductive layer 2-220 and electrically-conductive, reactive layer 2-240. The containment tube 2-117 can be included in the reactor vessel 2-110 if the electrically-conductive, reactive layer 2-240 is located on the outside of the catalytic tube 2-170, as illustrated. Alternatively, the containment tube 2-117 can be omitted from the reactor vessel 2-110 if the electrically-conductive, reactive layer 2-240 is located on the inside of the catalytic tube 2-170 (an order of layers reversed from the order shown in FIG. 2-2D).

For applications involving high-temperature operation, it is desirable to use a material for the support that has a very low coefficient of thermal expansion (CTE) or one that approximately matches a CTE for at least one layer, or for the combination of layers, deposited on the support 2-210. For example, the CTE of the support 2-210 can approximately match a CTE for at least one of, or the combination of, the electrically-conductive layer 2-220, the electrically-insulating layer 2-230, and the electrically-conductive, reactive layer 2-240. Differences in CTE values between the support 2-210 and layers may limit the length of the catalytic tubes 2-170 (e.g., due to cracking and/or delamination of the layers deposited on the support 2-210). For lower temperature operation (e.g., in domestic water heaters where the catalytic tubes 2-170 may operate at temperatures no greater than about 200° C. and some hydrogenation processes) design considerations relating to either or both of catalytic tube sag (described above) and differences in CTE values can be more relaxed. For example, larger CTE differences may be tolerated and/or a support 2-210 that may sag at 800° C. may operate fine at 200° C. without sagging.

For one example implementation, the support 2-210 is formed from alumina (CTE: 8.1×10⁻⁶/° C.) or austenitic stainless steel (CTE: 17.3×10⁻⁶/° C.), the electrically-conductive layer 2-220 is formed from copper (CTE: ˜16.4×10⁻⁶/° C.), the electrically-insulating layer 2-230 is formed from alumina, and the electrically-conductive, reactive layer 2-240 is formed from nickel (CTE: 13×10⁻⁶/° C.). Other material combinations are possible.

Referring again to FIG. 2-2B, the outer surface 2-218 of the support 2-210 (over at least a region on which the outer layers are deposited) can be machined and/or polished smooth so that subsequent layers deposited on the outer surface 2-218 will be smooth. The electrically-conductive layer 2-220, the electrically-insulating layer 2-230, and the electrically-conductive, reactive layer 2-240 can form a transmission line 2-270 suitable for propagating electrical excitation pulses (delivered by the electronic pulse driver 2-150) along the catalytic tube 2-170 which stimulate a catalytic reaction with the reactant gas or liquid that contacts the electrically-conductive, reactive layer 2-240 of the transmission line 2-270. Such a transmission line may be referred to as a “reactive transmission line” or “catalyzing transmission line.” When the support 2-210 is cylindrical, the transmission line 2-270 formed by the three outer layers is a coaxial transmission line, though other transmission line shapes can be used in other implementations. A smooth outer surface 2-218 of the support and of the electrically-insulating layer 2-230 can improve transmission line performance (e.g., reduce dispersion of excitation pulses propagating along the catalytic tube). A smooth and dense outer surface of the electrically-insulating layer can also reduce the dispersion of phonons generated in the electrically-conductive, reactive layer 2-240 and increase the reflection of phonons emanating from the electrically-conductive, reactive layer 2-240 back into the electrically-conductive, reactive layer 2-240 to improve catalytic reactions in the layer. In some implementations, the RMS roughness of the outer surface 2-218 can be between approximately or exactly 0.1 micron and approximately or exactly 1 micron, or between approximately or exactly 0.05 micron and approximately or exactly 0.5 micron. Preferably, the short pulse shape and high peak intensity should be maintained as the excitation pulse propagates along the catalytic tube(s) 2-170.

By making the outer surface 2-218 of the support 2-210 smooth, the deposited electrically-conductive layer 2-220 can be smooth to help reduce dispersion of excitation pulses propagating along the catalytic tube 2-170. The thickness of the electrically-conductive layer 2-220 can be between approximately or exactly 1 micron and approximately or exactly 500 microns, though thinner or thicker values may be used in some cases. The electrically-conductive layer 2-220 can be deposited by a physical deposition process (e.g., a plasma spray deposition or chemical vapor deposition) and/or by a plating process (e.g., electrochemically plated onto the support 2-210). According to some implementations, the electrically-conductive layer 2-220 is formed using multiple steps. First, the selected material (e.g., copper) is spray deposited in a reduction atmosphere to improve adhesion to the support 2-210. The spray deposition also can provide a porous morphology of the deposited layer to help accommodate differences in CTEs between the deposited layer and the support 2-210. In a next step, the same conductive material or a different conductive material can be plated onto the spray-deposited material to form a smooth skin through which electrical current will flow (driven by the pulse driver 2-150) when the system is in operation.

The electrically-insulating layer 2-230 can comprise a dielectric material, such as alumina, yttrium stabilized zirconia, or other dielectric material. Preferably, the dielectric material can withstand high operating temperatures (e.g., up to 800° C. for some applications) without incurring an appreciable change (e.g., more than 5%) in the dielectric constant or permittivity of the dielectric material. Alumina can withstand such high temperatures and maintain its dielectric constant to within about 5% between room temperature and high temperature operation up to 800° C. The electrically-insulating layer 2-230 can be spray coated in particulate form onto the electrically-conductive layer 2-220. The spray deposition can provide some porosity of the deposited layer (to accommodate differences in CTE values between the electrically-insulating layer 2-230 and adjacent layers. The thickness of the electrically-insulating layer 2-230 can be between approximately or exactly 100 microns and approximately or exactly 400 microns, though thinner or thicker values may be used in some cases.

The reactive layer 2-240 can be deposited onto the electrically-insulating layer 2-230 using a physical deposition process (e.g., sputtering, plasma spray deposition, chemical vapor deposition, application of a powder form of the material and sintering) and/or an electroplating process. This outer layer comprises a reactive layer where heat is generated during operation of the reactor vessel. The material for the reactive layer 2-240 is chosen to catalyze a reaction with the reactant substance or substances that contact(s) the reactive layer 2-240. In some implementations, atoms of the reactant substance (such as hydrogen atoms or deuterium atoms) can enter and diffuse into the lattice of the reactive layer 2-240. Example materials for the reactive layer 2-240 include, but are not limited to, nickel (Ni), copper (Cu), palladium (Pd), platinum (Pt), rhodium (Rd), titanium (Ti), tungsten (W), cobalt (Co), iron (Fe), any transition metal that can absorb hydrogen, or some combination these materials formed in an alloy and/or multilayer structure. In some implementations, the reactive layer 2-240 can be formed as a multilayer structure (e.g., a thin, micron or sub-micron thick layer of Pd deposited over Ni). Such a multilayer structure may improve absorption of hydrogen into nickel.

In an exemplary implementation, Ni used. Nickel has several desirable features which include low cost and suitability for physical deposition processes. Another desirable feature of Ni is that absorption of hydrogen within the material increases when the temperature of the material increases, which is an opposite trait than absorption of hydrogen in Pd. In some implementations, the deposited nickel can be treated to form a porous or so-called “grainy nickel” which can provide a more reactive surface and facilitate ingress of atoms from the reactant substance into the reactive layer 2-240.

The thickness of the reactive layer 2-240 can be between approximately or exactly 200 micron and approximately or exactly 900 microns, though thicker values may be used in some cases. The reactive layer 2-240 can be deposited to a thickness that is sufficient to dissipate heat generated from catalyzed reactions within the layer without damage to the layer. As such, the thickness of the reactive layer 2-240 may be selected based on a peak temperature expected for a particular application. An application for producing domestic hot water or for a hydrogenation process may operate at a lower temperature than an application for heating systems to heat a residential or commercial building and therefore have catalytic tubes 2-170 with thinner reactive layers (e.g., no greater than 200 microns).

A countervailing consideration for the thickness of the reactive layer 2-240 is diffusion of atoms from the reactant gas or liquid into the reactive layer. Preferably, the reactive layer 2-240 is thin enough so that a sufficient supply of atoms (e.g., hydrogen atoms) can diffuse into the reactive layer 2-240 to an active portion of the reactive layer. During operation of the system 2-100, the reactive layer 2-240 is exposed to both the reactant which flows across the outer surface of the reactive layer 2-240 due to the reactant processing system 2-140 and to electrical current from broad-spectrum excitation pulses delivered by the electronic pulse driver 2-150. Because of the skin effect, the current flowing through the reactive layer 2-240 can be confined within a short distance δ from the interface between the electrically-insulating layer 2-230 and the reactive layer 2-240, as depicted in FIG. 2-2E. This short distance δ is a region within which catalyzed reactions can be highest and is part of the active portion of the reactive layer 2-240.

The distance δ, known as the skin depth, is determined in part by the frequency (or frequency spectrum) of the excitation pulse driven along the transmission line 2-270 by the pulse driver 2-150 and the resistivity of the material used to form the reactive layer 2-240.

Both the frequency spectrum and resistivity can be tailored, to some extent, during design and/or operation of the system. For example, the excitation pulse duration can be tailored during design of the pulse driver 2-150 and may be adjustable by a user during operation of the system in some implementations of the pulse driver. The pulse amplitude (and therefore pulse energy and average power in a train of pulses) may also be adjustable by a user during operation of the system. The resistivity of the reactive layer 2-240 can be tailored by alloying metals and/or by selecting or adjusting deposition processes during design and manufacture of the catalytic tubes 2-170, for example.

For some hydrogenation processes, short excitation pulses may not be required, and DC currents may be sufficient to stimulate, turn on, and turn off the hydrogenation process. For DC currents and longer pulses, skin depth of the applied current is less of a concern or no concern and the reactive layer 2-240 can be thicker than 900 microns.

To obtain uniform layer thicknesses for each of the three layers 2-220, 2-230, 2-240 during a physical deposition process, the support 2-210 can be rotated (about its central axis along the z direction) and may further be translated within a deposition chamber.

During operation of the system with short pulses, most of the current from the excitation pulses provided by the pulse driver 2-150 flows within the region bound by the skin depth δ. In this region, the driven current can excite phonons in the reactive layer 2-240. The phonons can propagate throughout the reactive layer 2-240 dissipating energy to the lattice as they propagate. These phonons have their highest energy in the region of the reactive layer 2-240 bound by the skin depth and they aid in stimulating reactions of the reactant gas or liquid catalyzed by the reactive layer 2-240. The active portion of the reactive layer 2-240 can extend beyond the skin depth since phonons can propagate beyond the skin depth. The catalyzed reactions produce heat in the reactive layer 2-240 that, in part, radiates outward to the surrounding containment tube 2-117 where the heat can thermally couple to the heat-transfer substance in the reactor vessel 2-110.

At the ends of the catalytic tube 2-170, along portions of the catalytic tube that extends outside the reactor vessel shell 2-119, the electrically-conductive layer 2-220 can be exposed along a first length L₁ of the support 2-210 (in the ±z directions of the drawing for FIG. 2-2A), so that an electrical connection can be made to the electrically-conductive layer 2-220. Similarly, the reactive layer 2-240 can also be exposed along at least a second length L₂ of the support 2-210 so that an electrical connection can be made to that layer. The electrically-insulating layer 2-230 may or may not be exposed along a length of the support 2-210.

Other implementations of a catalytic structure are also possible, and a tube shape is not necessarily required. In some cases, the catalytic structure can be implemented as a planar transmission line, or as a plurality of strip transmission lines running in parallel. FIG. 2-2F depicts an example of a catalytic structure that comprises a plurality of strip transmission lines 2-272 that can run in parallel along a substrate 2-222. The assembly can extend through the reactor vessel 2-110. The substrate could be a metal substrate (e.g., copper) which can provide a ground plane and thermal coupling to a heat-transfer substance flowing through the reactor vessel 2-110. The heat-transfer substance may or may not contact a back surface 2-225 of the substrate. The strip transmission lines 2-272 can be patterned on the substrate using planar fabrication processes (e.g., depositing the electrically-insulating layer 2-230, depositing the reactive layer 2-240, patterning and etching the strip transmission lines 2-272). The width of each strip transmission line 2-272 can be between 0.2 mm and 4 mm. In some implementations, the strip transmission lines 2-272 can be enclosed by a cover 2-290 to form a reaction chamber 2-250 through which the reactant can flow. The cover 2-290 may or may not be electrically conductive.

In some cases, the cover 2-290 can be a conductive metal (e.g., copper) that electrically connects to a conductive substrate 2-222 to provide a ground reference all around the strips formed from the reactive layer 2-240. With a ground reference all around the strips and approximately equal distances from the strips, the skin effect (indicated by the dashed lines) can occur on both sides of the strips. The added skin effect on the upper surfaces of the strips formed from the reactive layer provide a shorter path for reactant atoms to enter into active regions of the reactive layer 2-240. The conductive cover 2-290 can also provide thermal coupling to a heat-transfer substance, which may or may not contact the exterior of the conductive cover 2-290.

### 2-3. Feed-Thru and Support of Catalytic Tubes

FIG. 2-3 depicts, in a cross-sectional view, further details of a portion of the system of FIG. 2-1A where the containment tube 2-117 passes through a feed-thru manifold 2-114 located at one end of the reactor vessel 2-110. Visible in the drawing are an output heat-transfer port 2-116 and a reactant output port 2-115 (located behind one of the containment tubes 2-117). The cross-section passes centrally through the output heat-transfer port 2-116, the reactor vessel 2-110, and two of the containment tubes 2-117. The feed-thru structure supports the containment tubes 2-117, which each house and support a catalytic tube 2-170 (not installed in the drawing of FIG. 2-3).

The feed-thru manifold 2-114 comprises a manifold chamber 2-310 to contain and distribute the reactant(s) to each containment tube 2-117. The reactant(s) can enter each containment tube 2-117 tube through a hole 2-312 in the sidewall of the containment tube within the manifold chamber 2-310. The reactant(s) can then travel along a sheath within the reaction chamber 2-250 (FIG. 2-2C) between an inner wall of the containment tube 2-117 and the reactive layer 2-240 on the outer surface of the catalytic tube 2-170 (which is installed within the containment tube 2-117 when the system is operating). The reactant(s) can exit a similar hole 2-312 in the sidewall of the containment tube 2-117 within the second feed-thru manifold 2-114 at an opposite end of the reactor vessel 2-110.

Though the manifolds 2-114 and support structure for the containment tubes 2-117 are integrated together and attached to the end of the reactor vessel 2-110 in the example reactor vessel, the manifold structure could be located elsewhere outside the reactor vessel.

For example, a manifold could be mounted apart from the reactor vessel and individual lines can run from the manifold to each containment tube 2-117. In such implementations, the support structure for the containment tubes 2-117 could be located on end walls of the vessel chamber 2-360.

For the example implementation of FIG. 2-3, the containment tubes 2-117 pass through and are sealed to end walls 2-320a, 2-320b of the reactor vessel 2-110 and the feed-thru manifold 2-114, respectively so that the manifold chamber 2-310 is airtight. The end wall 2-320a of the reactor vessel 2-110 can be part of the outer shell 2-119 for the reactor vessel 2-110. Sealing of the containment tubes 2-117 to the end walls 2-320a, 2-320b can be done using high temperature adhesives, solder, brazing, welding, Teflon® or silicone O-rings, or some combination thereof, for example. The containment tubes 2-117 can be formed from stainless steel, for example. Other materials that can be used to form the containment tubes include aluminum, copper, bronze, brass, metal alloys comprising one or more of the preceding materials, galvanized steel, steel coated to resist corrosion.

The feed-thru manifold 2-114 can be formed, according to some implementations, by attaching a cup-shaped end-piece 2-350 to an end of the reactor vessel shell 2-119. The attachment of the end-piece 2-350 can be done by fasteners (along with a gasket or O-ring) or by an adhesive, solder, brazing, or welding, for example. Prior to attaching the end-piece 2-350, the containment tubes 2-117 can be installed in the reactor vessel 2-110 and sealed to the end walls 2-320a of the reactor vessel. The material used to make the feed-thru manifold 2-114 and the reactor vessel shell 2-119 can comprise stainless steel and/or other materials such as aluminum, copper, bronze, brass, metal alloys comprising one or more of the preceding materials, galvanized steel, steel coated to resist corrosion.

According to some implementations, the containment tube 2-117 comprises a tubular portion 2-317 and an insert 2-373. A portion of the insert 2-373 fits within the tubular portion (located inside the manifold chamber 2-310 in the example implementation). The fit of the insert 2-373 can be a press fit or friction fit to aid in sealing the two pieces together. In some cases, the insert 2-373 can be soldered, brazed, or welded to the tubular portion 2-317. Since the joint between the insert 2-373 and tubular portion can be located within the manifold chamber 2-310, a leak at the joint would not affect system operation appreciably. During assembly of the feed-thru manifold 2-114, the tubular portion 2-317 can be installed in the vessel chamber 2-360 and sealed to end walls 2-320a of the reactor vessel 2-110 to prevent influx of heat-transfer substance in the vessel chamber 2-360 into the containment tube 2-117. The inserts 2-373 can then be installed in the tubular portions 2-317 and the end-piece 2-350 attached. The inserts 2-373 can then be sealed to the end wall 2-320b of the feed-thru manifold 2-114.

The inserts 2-373 can be threaded at one end, similar to a Swagelok® fitting. A nut 2-370 can thread onto the insert 2-373 and engage (press on) a ferule 2-375 when tightened onto the insert 2-373. The ferule 2-375 can close inward, grabbing and supporting the catalytic tube 2-170 within the containment tube 2-117. The nut 2-370 and ferule 2-375 can facilitate installation and replacement of the catalytic tube 2-170.

In some cases, the ferule 2-375 can seal the gas chamber formed as an annular reaction chamber 2-250 (FIG. 2-2A) between the catalytic tube 2-170 (when installed within the containment tube) and the containment tube 2-117. In other cases, an O-ring can be installed between the catalytic tube 2-170 and containment tube 2-117 at each end of the catalytic tube 2-170 to form a sealed gas chamber comprising an annular reaction chamber 2-250 around the catalytic tube 2-170. In some implementations, an insulating sleeve (not shown) can be placed over the catalytic tube 2-170, so that the sleeve is located between the catalytic tube 2-170 and the ferule 2-375. The insulating sleeve can provide electrical isolation of the reactive layer 2-240 from the containment tube 2-117 and also assist in forming a gas-tight seal at the ends of the containment tube 2-117. In some cases, the ferule 2-375 can be formed from an electrically insulating material (such as Teflon® or silicone rubber) to electrically isolate the catalytic tube 2-170 and its transmission line 2-270 from other metals in the reactor vessel 2-110. Because the ferule 2-375 can be formed from a polymer, it can grip the catalytic tube 2-170 without deforming the tube to facilitate removal and replacement of the catalytic tube 2-170.

The reactor vessel 2-110 comprises a vessel chamber 2-360 through which a heat-transfer liquid or gas can flow. The heat-transfer substance comes into contact with the containment tubes 2-117 which receive heat from catalytic tubes 2-170 (not shown in FIG. 2-3) (or provide heat to the catalytic tubes for some hydrogenation applications) when the catalytic tubes 2-170 are installed within the containment tubes 2-117 and the system is operating. The vessel chamber 2-360 can include a plurality of baffles 2-363 located throughout the vessel chamber 2-360 to aid in mixing the heat-transfer liquid or gas as it flows through the chamber 2-360. The heat-transfer liquid or gas enters the vessel chamber 2-360 through the one or more input heat-transfer ports 2-112 and flows out the one or more output heat-transfer ports 2-116. The inner diameter D of the vessel chamber can be between approximately or exactly 10 cm and approximately or exactly 80 cm, though larger diameters are possible to scale heat production or hydrogenation. With larger diameters, more containment tubes 2-117 and catalytic tubes 2-170 can be installed in the reactor vessel 2-110, increasing its thermal output or surface area for hydrogenation.

### 2-4. Electrical Connections to the Catalytic Tubes

FIG. 2-4A depicts details of an electrical connector 2-400 that can attach near an end of the catalytic tube 2-170 of FIG. 2-2A when the tube is installed in the reactor vessel 2-110 of FIG. 2-1A. The illustration is a cross-section taken through the catalytic tube 2-170 and electrical connector 2-400. The electrical connector 2-400 is used to make electrical connections to the electrically-conductive layer 2-220 and the reactive layer 2-240 which form the transmission line 2-270 along the catalytic tube 2-170. The electrical connector 2-400 comprises a clamp 2-405 and a coupling PCB 2-180a (or coupling PCB 2-180b) retained within the clamp 2-405. The electrical connector 2-400 is one example of apparatus that can be used to make the electrical connections to the transmission line 2-270. Other structures of apparatus to make the electrical connections to the transmission line 2-270 are also possible.

The clamp 2-405 comprises at least two clamping plates located on opposites sides of the coupling PCB 2-180. A first clamping plate 2-410 can be located on a first side of the coupling PCB 2-180 and can be configured to press on a first collet 2-415 when drawn toward a second clamping plate 2-420. The second clamping plate 2-420 can be located on a second, opposing side of the coupling PCB 2-180 and be configured to press on a second collet 2-425 when the second clamping plate 2-420 is drawn toward the first clamping plate 2-410. The clamp 2-405 can further comprise a backing plate 2-430, which may or may not be electrically conducting. The backing plate 2-430 can have threaded holes 2-462 to engage the screws 2-460 and draw the first clamping plate 2-410, PCB 2-180, and second clamping plate 2-420 toward the backing plate. The first clamping plate 2-410 and the second clamping plate 2-420 can be formed from a non-conducting material, such as a polymer.

The coupling PCB 2-180 can include at least one transmission line 2-185, depicted in FIG. 2-4B, to carry electrical excitation pulses provided by the pulse driver 2-150. The transmission line 2-185 can comprise two conductive traces running parallel to each other and separated by an insulating layer of the coupling PCB 2-180 (stacked one above the other in the plan view of FIG. 2-4B). The conductive traces of the transmission line 2-185 can connect to annular contacts 2-188 located on opposing surfaces of the coupling PCB 2-180 (also stacked one above the other in the drawing). However, contacts of shapes other than those shown in FIG. 2-4B can be used. The annular contacts 2-188, or contacts of other shapes, can be located on the coupling PCB 2-180 for making electrical connections to the first collet 2-415 and the second collet 2-425.

Electrical connection between a first conductive trace of the transmission line 2-185 on a first side 2-182 of the coupling PCB 2-180 and the electrically-conductive layer 2-220 of the catalytic tube can be made with the first collet 2-415 and a first conductive sleeve 2-452. The first conductive sleeve 2-452 can be placed onto the catalytic tube 2-170 at a location where the electrically-conductive layer 2-220 is exposed (along length L₁, see FIG. 2-2A) and soldered or otherwise electrically connected to the electrically-conductive layer 2-220. The first conductive sleeve 2-452 can comprise a highly conductive metal such as copper, aluminum, gold, or an alloy or combination thereof. The first collet 2-415 can be placed over the first conductive sleeve 2-452. When the clamp 2-405 is tightened (by tightening screws 2-460, for example), drawing the first clamping plate 2-410 toward the second clamping plate 2-420, an end face 2-417 of the first collet 2-415 engages with a contact (e.g., annular contact 2-188) on the coupling PCB 2-180 making an electrical connection between the first conductive trace of the transmission line 2-185 and the first collet 2-415. The contact on the PCB can be annular in shape as shown in FIG. 2-4B or can be semi-annular in shape extending at least part way around the catalytic tube 2-170, for example. In some implementations, an area of the contact on the coupling PCB 2-180 is the same size as the end face 2-417 of the first collet 2-415 that comes into contact with the contact pad or electrical trace.

When the clamp 2-405 is tightened further, the first non-conducting clamping plate 2-410 squeezes the first collet 2-415 due to a mating conical hole 2-413 in the clamping plate 2-410. The conical hole 2-413 presses on the conical outer wall of the first collet 2-415 and tightens the first collet 2-415 onto the first conductive sleeve 2-452, making electrical connections between the first collet 2-415, the first conductive sleeve 2-452, and the electrically-conductive layer 2-220 on the catalytic tube 2-170. The taper angle α of the collet outer walls and mating conical holes in the electrical connector 2-400 can be between 5 degrees and 45 degrees, as measured between a line running along the outer angled wall of the collet (in the z direction) and a line running in the z direction along the inner wall of the collet (roughly indicated above the drawing of the electrical connector 2-400).

A similar assembly can be used to make electrical connection between a second conductive trace of the transmission line 2-185 on a second side 2-184 of the coupling PCB 2-180 and the reactive layer 2-240 on the catalytic tube 2-170 with the second clamping plate 2-420 and second collet 2-425. Tightening the screws 2-460 also pushes an end face 2-427 of the second collet 2-425 into electrical contact with the second trace on the second side 2-184 of the coupling PCB 2-180 and tightens the second collet 2-425 onto a second conductive sleeve 2-454 placed over the reactive layer 2-240.

The electrical connections via the collets and conductive sleeves to the ends of the transmission line 2-270 of the catalytic tube 2-170 can be very low impedance (e.g., from 0.001 milliohm to 2 milliohms) and the pair may be designed to match the RF impedance of the transmission line 2-270 of the catalytic tube 2-170, which can have a value in a range from 0.1 ohm to 75 ohms or from 0.1 ohm to 25 ohms in some cases. Additionally, transmission lines formed on the coupling PCB 2-180 can be designed to have an impedance that matches the impedance of the transmission line 2-270 of the catalytic tube 2-170. In this manner, catalytic tubes 2-170 can be electrically connected in series with matched impedances. As such, power reflections to the pulse driver 2-150 are reduced to levels that can be tolerated by electrical components in the pulse driver 2-150 and power coupling into the catalytic tubes 2-170 is increased. Further, fidelity and frequency content of the electrical excitation pulses can be maintained. A matched terminating impedance can be located at an end of the total electrical path after one or more connected catalytic tubes 2-170 to further prevent power reflections and pulse distortion.

According to some implementations, the first collet 2-415 is identical to the second collet 2-425 in size, shape, and dimensions. This can be possible even though the outer diameter of the catalytic tube 2-170 can be appreciably different where the two collets are located (see FIG. 2-2A). The first conductive sleeve 2-452 and the second conductive sleeve 2-454 can accommodate the differences in diameters. For example, both conductive sleeves can have the same outer diameter that fits within the two collets. The inner diameter of the first conductive sleeve 2-452 can be smaller than the inner diameter of the second conductive sleeve 2-454 to accommodate the differences in diameters of the catalytic tube 2-170 where the exposed electrically-conductive layer 2-220 is located along length L₁ and the exposed reactive layer 2-240 is located along length L₂. In some implementations, the first collet 2-415, the second collet 2-425, the first conductive sleeve 2-452, and the second conductive sleeve 2-454 can be plated with a high conductor, such as gold or copper, to reduce contact resistance and provide better impedance matching. Further, the exposed portions of the electrically-conductive layer 2-220 and the reactive layer 2-240 can be plated with a high conductor, such as gold or copper.

The backing plate 2-430 can include one or more resilient contact elements for applying a resilient force against the second clamping plate 2-420. A resilient contact element can comprise at least one spring, at least one flexural tab, at least one wave washer, at least one piece of compliant material, etc. In the illustration of FIG. 2-4A, the resilient contact element comprises a wave washer 2-435 that can resiliently press against the second clamping plate 2-420 at three or more contact points. The resilient contact element can allow for expansion and contraction of the length and diameter of the catalytic tube 2-170 due to changes in operating temperatures while maintaining sufficient and nearly constant pressure on the first collet 2-415 and the second collet 2-425 to preserve the integrity of electrical connections to the electrically-conductive layer 2-220 and the reactive layer 2-240.

The clamp 2-405 may or may not include counter springs 2-440, which can be located anywhere between the first clamping plate 2-410 and PCB 2-180 and/or between the second clamping plate 2-420 and PCB 2-180. The counter springs 2-440 are arranged to push apart the first clamping plate 2-410 and the second clamping plate 2-420 away from the PCB 2-180 when screws 2-460 are loosened. The counter springs 2-440 can facilitate removal of the electrical connector 2-400 from the catalytic tube 2-170 (e.g., for replacement of the catalytic tube).

Although FIG. 2-4A illustrates only one electrical connector 2-400 for one catalytic tube 2-170, other configurations are possible. In some cases, the coupling PCB 2-180 can be large enough to extend across the ends of two or more catalytic tubes mounted in a reactor vessel 2-110. Transmission lines formed on the coupling PCB 2-180 can make electrical connections between different catalytic tubes 2-170. For example, the coupling PCB 2-180 can have transmission lines 2-154 (FIG. 2-1A) formed thereon to convey excitation pulses from the pulse driver 2-150 and from catalytic tube to catalytic tube. The PCB can connect catalytic tubes 2-170 in serial or in parallel. Parallel connections may require higher current output from the pulse driver 2-150 whereas serial connections may require higher voltage output from the current driver compared to a single catalytic tube. In some implementations, the first clamping plate 2-410, second clamping plate 2-420, and backing plate 2-430 can also extend across the ends of two or more catalytic tubes mounted in a reactor vessel 2-110. Extending the clamping plates and backing plate can reduce piece count, expedite assembly, and allow for a tighter packing of containment tubes 2-117 and catalytic tubes 2-170 in a reactor vessel at the cost of tighter alignment tolerances for distances between catalytic tube tubes. Having separate clamping plates and backing plates for each catalytic tube or subgroups of tubes may provide higher assurance of adequate electrical connections to the electrically-conductive layer 2-220 and the reactive layer 2-240 on each catalytic tube 2-170.

FIG. 2-4C illustrates, in a cross-sectional view, further details of the first collet 2-415 and the second collet 2-425 that are used to make electrical connections between transmission lines 2-185 on the coupling PCB 2-180 and the transmission line 2-270 of the catalytic tube 2-170 with the electrical connector 2-400 of FIG. 2-4A. FIG. 2-4D is a cross-section, perspective view of the second collet 2-425, which can be identical to the first collet 2-415, though different collets can be used in some cases. The first collet 2-415 and the second collet 2-425 each comprise longitudinal cuts 2-422 extending part way along the length of each collet. The longitudinal cuts 2-422 allow each collet to flex and its inner diameter to reduce when the conical outer wall of each collet is pressed on by the mating conical holes 2-413 in the first clamping plate 2-410 and the second clamping plate 2-420.

FIG. 2-4E depicts an end of a catalytic tube 2-170 that can be inserted into the electrical connector 2-400 of FIG. 2-4A. The first conductive sleeve 2-452 and the second conductive sleeve 2-454 are placed over exposed regions of the electrically-conductive layer 2-220 and the reactive layer 2-240, respectively, on each catalytic tube 2-170. In some implementations, the second conductive sleeve 2-454 can include a flange 2-457 at one end to act as a stop for the second collet 2-425 when installing the clamping plates 2-410, 2-420 and coupling PCB 2-180.

In some implementations, one of the first collet 2-415 and second collet 2-425 may or may not include cuts for flexural purposes, may not have a conical shape, and a conductive sleeve may not be used. Instead, the first collet or second collet can have an end face 2-417 or 2-427 and be placed over and clamped to the exposed conductive layer. The collet may have one through-cut to allow expansion and compression of the collet ring, or the collet may be formed as two half pieces that can be clamped to either side of the catalytic tube 2-170. In some cases, the collet may be formed as multiple pieces that clamp to the catalytic tube 2-170. For easier implementation, the collet closer to the center of the catalytic tube (the second collet 2-425 in FIG. 2-4A) can be clamped to the tube. The electrical connector 2-400 can then include one clamping plate that only grasps the clamped collet and, for example, the first clamping plate 2-410 and first collet 2-415 as shown in FIG. 2-4A. When the first clamping plate 2-410 is drawn toward the clamping plate that grasps the clamped collet, the first clamping plate 2-410 will engage the first collet and draw it and the PCB 2-180 toward the clamped collet establishing electrical contact between the two collets and their respective annular contacts 2-188 on the PCB 2-180.

### 2-5. Additional Reactor Vessel Implementations

FIG. 2-5 depicts another implementation of a reactor vessel 2-510 that can be used in the system of FIG. 2-1A. The illustration is a cross-section that passes through the containment tube 2-117. Electrical connections to the catalytic tube 2-170 are not shown to simplify the drawing. The reactor vessel 2-510 shows only one containment tube 2-117 and catalytic tube 2-170 within the reactor vessel, but more containment tubes and catalytic tubes can be mounted within such a reactor vessel.

The reactor vessel 2-510 can be supplied with a reactant or reactants through at least one reactant input line 2-144 and supplied with a heat-transfer liquid or gas through at least one heat-transfer line 2-164. The reactant(s) and heat-transfer gas or liquid can exit through at least one reactant output port and at least one output heat-transfer port, respectively, at an opposite end of the reactor vessel 2-510. The reactor vessel 2-510 comprises a vessel chamber 2-520, which can be placed under vacuum, surrounding the containment tube 2-117 and a water jacket 2-530. Vacuum can be established within the vessel chamber 2-520 through vacuum port 2-522. The vessel chamber 2-520 can reduce heat transfer from the catalytic tube 2-170 to an external environment.

The reactor vessel 2-510 comprises an outer shell 2-119 containing a water jacket 2-530 surrounding the containment tube 2-117. The water jacket 2-530 comprises a jacket tube 2-535 having a larger inner diameter than the outer diameter of the containment tube 2-117 so that a sheath of heat-transfer fluid or gas can flow in a space between the containment tube 2-117 (from which heat is extracted) and the jacket tube 2-535. The jacket tube 2-535 can be part of the outer shell 2-119 which forms the vessel chamber 2-520. Similar to the reactor vessel 2-110 of FIG. 2-1B and FIG. 2-3, the reactant(s) can flow in a sheath along a reaction chamber 2-250 between an outer surface of the catalytic tube 2-170 and an inner surface of the containment tube 2-117. This reaction chamber 2-250 can be sealed at each end of the reactor vessel by a seal 2-552 (such as an O-ring).

For the reactor vessel designs of FIG. 2-2C and FIG. 2-5, the reaction chamber 2-250 can be packed with a material having high thermal conductivity (e.g., over 10 W m⁻¹ K⁻¹) and providing electrical isolation within the reactor vessel along an active length of the catalytic tube 2-170 where heat is generated. An example of a material having high thermal conductivity is alumina, which can also provide electrical isolation, though other electrically insulating materials having a high thermal conductivity may be used. To impede thermal conductivity near the ends of the catalytic tube 2-170, where the seal 2-552 and electrical connections are located, the reaction chamber can be packed with an electrically-insulating fill material that has a low thermal conductivity, such as silica, though other materials having a low thermal conductivity may be used.

### 2-6. Drive Electronics

FIG. 2-6A and FIG. 2-6B depict an example implementation of drive electronics 2-600 used to transmit excitation pulses down the transmission line 2-270 of the catalytic tubes 2-170. The drive electronics 2-600 can comprise part or all of the pulse driver 2-150. In the illustrations, the drive electronics 2-600 are implemented on several printed circuit boards (e.g., the coupling PCB 2-180a and one or more auxiliary PCBs 2-621, 2-622, 2-623 that electrically couple to the coupling PCB 2-180a). Implementing at least some of the drive electronics 2-600 on the coupling PCB 2-180a can improve the quality of the excitation pulses delivered to the catalytic tube(s) 2-170. For example, shorter pulse rise times and/or durations may be possible compared to generating the excitation pulses with a remote electronic driver and transmitting the excitation pulses over lengthy transmission lines to the catalytic tube(s) 2-170.

In some implementations, the drive electronics 2-600 are configured to receive pulses from a pulse source (which can be located separately from the drive electronics) and adapt the received pulses for delivery as excitation pulses to the electrical connector 2-400 and transmission line 2-270 of the catalytic tube 2-170. Adaptation of the received pulses can comprise at least one of (1) preparing the excitation pulses to have a selected voltage amplitude, (2) providing sufficient current for each excitation pulse, (3) preparing the excitation pulses to have a selected pulse shape, and providing the excitation pulses over a transmission line that is impedance matched to the electrical connector 2-400 and transmission line 2-270.

In some implementations, the pulses received by the drive electronics 2-600 can be optical pulses. The optical pulses can be converted to electrical excitation pulses by the drive electronics 2-600 (with an opto-isolator, for example). Using such optical isolation can protect the pulse source from electromagnetic interference generated by the drive electronics 2-600 when outputting excitation pulses to the catalytic tube(s) 2-170.

The drive electronics 2-600 can comprise high-power, high-speed transistors 2-630 (such as gallium-nitride transistors) to perform voltage switching at moderate voltages (e.g., up to 100 volts) with high currents for generation of the excitation pulses. The transistors 2-630 (visible in FIG. 2-6B) can be mounted on the coupling PCB 2-180a in close proximity (e.g., within about 10 cm) to the location where the electrical connector 2-400 contacts the coupling PCB 2-180a. These transistors can generate significant heat during operation of the system. A heat sink 2-610 can be thermally coupled to the transistors 2-630 to aid in dissipating the heat generated by the transistors. The heat sink 2-610 can have a channel within it to flow a coolant through the heat sink. The coolant can be circulated through the heat sink with coolant lines 2-612. There can be a back plate 2-611 mounted to an opposite side of the coupling PCB 2-180a (visible in FIG. 2-6B) for additional cooling and to aid in connecting and thermally coupling the heat sink 2-610 to the coupling PCB 2-180a. The heat sink 2-610 and the back plate 2-611 can be made from a material having high thermal conductivity, such as copper or aluminum.

The opposite end of the catalytic tube 2-170 from the near end shown in FIG. 2-6A can connect to a different coupling PCB 2-180b (FIG. 2-1A) which may not include drive electronics 2-600. The coupling PCB 2-180b can include transmission lines to couple the distal end of a first catalytic tube 2-170 to the distal end of a second catalytic tube 2-170 at the same end of the reactor vessel 2-110. The coupling PCB 2-180b can also include probe points and may further include sensing electronics for detecting excitation pulses which have passed through a catalytic tube 2-170 (e.g., to check excitation pulse quality and evaluate tube performance). Output from the sensing electronics can be provided to the system controller 2-190 to monitor operation of the system. The system controller 2-190 can output a signal or shut down the system if the system controller 2-190 determines that system service is needed (e.g., detection of a faulty catalytic tube 2-170).

Each of the coupling PCBs 2-180a, 2-180b can include transmission lines patterned on the PCB to carry pulses to and from the transmission lines formed on the catalytic tubes 2-170. The transmission lines patterned on the coupling PCBs 2-180a, 2-180b can be engineered to have RF impedance values that match the RF impedance values of the transmission lines formed on the catalytic tubes 2-170. For example, the RF impedance values of the transmission lines patterned on the coupling PCBs 2-180a, 2-180b be within 10% of the RF impedance values of the transmission lines formed on the catalytic tubes 2-170 over a range of frequencies from approximately or exactly 250 MHz to 2 GHz, impedance matching over other frequency ranges is possible.

### 3. Example Hydrogenation Applications

The above-described systems 1-100, 2-100 for catalyzing hydrogenation reactions in reactor vessels 1-102, 2-110, 2-510 can be used in a wide variety of applications, some of which are described in this section. In some implementations, the systems and methods can be used to perform full hydrogenation of a product (i.e., converting all double bonds in an unsaturated compound to single bonds, resulting in a saturated compound). In some implementations, the systems and methods can be used to perform partial hydrogenation of a product (i.e., converting only some of the double bonds in an unsaturated compound (also referred to as an unsaturated reactant) to single bonds, leaving a mix of saturated and unsaturated compounds in the final product).

The hydrogenation reactions implemented with the above-described systems and methods can include hydrogenation of organic molecules and inorganic molecules for some applications. Hydrogenation is most commonly associated with organic compounds, particularly in the food, pharmaceutical, and chemical industries. Hydrogenation of alkenes converts them into alkanes by adding hydrogen across the double bond. Alkynes can be hydrogenated to alkenes or fully to alkanes, depending on the reaction conditions. Aromatic compounds like benzene can be hydrogenated to cyclohexane, though this can requires strict operating conditions. Aldehydes and ketones can be hydrogenated to produce primary and secondary alcohols, respectively. Nitro groups (—NO₂) can be hydrogenated to amines (—NH₂), which is useful in the production of aniline and related compounds. Hydrogenation of unsaturated fats converts them into saturated fats, used in food products like margarine and shortening.

In some applications, the hydrogenation reactions can include hydrogenation of inorganic molecules, though these reactions may operate at higher temperatures and pressures than used for hydrogenation of organic molecules. Such applications may be pertinent to energy storage, environmental applications, and catalysis. For example, certain metal oxides can be reduced to metals through hydrogenation, important in metallurgy and materials science. As an example, tungsten oxide can be reduced to tungsten metal using hydrogenation. Hydrogenation of CO₂ to form methane (methanation) or methanol is an area of significant interest for carbon capture and utilization, as well as for producing synthetic fuels. Hydrogenation of nitrogen oxides (NOₓ) to produce ammonia (NH₃) or other nitrogen compounds is used in environmental applications to reduce harmful emissions. Certain metal hydrides and other materials can undergo reversible hydrogenation-dehydrogenation cycles, which are useful for hydrogen storage technologies.

As mentioned above, the systems and methods can be used for hydrogenation reactions in the food processing industry, petrochemical and fuel production industries, and chemical manufacturing processes. In the food industry, hydrogenation can be used to convert vegetable oils (unsaturated reactants) into semi-solid or solid fats, such as margarine and shortening. This process can improve texture, stability, and shelf life of the produced food items.

For petrochemical manufacture and fuel industries, hydrogenation can be used to refine crude oil (an unsaturated reactant) into various products, such as gasoline and diesel, by reducing the content of unsaturated hydrocarbons. In other applications, the hydrogenation reactions can be used to convert carbon dioxide (an unsaturated reactant) into methane or methanol, which can be used as fuels or chemical feedstocks, aiding in carbon capture and utilization efforts. Hydrogenation of bio-oils (unsaturated reactants) derived from biomass can be performed with the above-described systems and methods to produce renewable diesel and jet fuels. Hydrogenation can be used to remove sulfur from petroleum products, producing cleaner fuels and reducing sulfur emissions. Hydrogenation can be used to reduce aromatic compounds in fuels and lubricants to improve the quality and stability of the fuels and lubricants. Hydrogenation processes may be used in some cases for hydrogen fuel storage or hydrogen fuel production. For example, some materials may be hydrogenated and/or dehydrogenated to store and/or release hydrogen efficiently. In one example, ammonia or water can be dehydrogenated in a catalytic process to release hydrogen fuel and nitrogen gas or oxygen gas, respectively.

An array of different applications exist in the chemical and textile manufacturing industries to which the systems and methods described above can be applied. These applications include, but are not limited to, making different chemical compounds in liquid or solid form, polymers, pharmaceuticals, improving durability of polymers and rubbers, and making agricultural chemicals. Hydrogenation of aldehydes and ketones may be performed with the above-described systems and methods to produce alcohols. Hydrogenation of nitriles, nitro compounds, and imines may be performed with the above-described systems and methods to produce primary, secondary, and tertiary amines. Hydrogenation with the above-described systems and methods may be used to improve the stability and performance of certain polymers, such as converting polybutadiene to polybutylene, which is more stable and less reactive. Hydrogenation of synthetic rubbers with the above-described systems and methods may be used to enhance their resistance to heat, oxidation, and other environmental factors. Hydrogenation processes with the above-described systems and methods can be used to modify the properties of nanomaterials, such as carbon nanotubes and graphene, for various applications in electronics, catalysis, and materials science. Hydrogenation with the above-described systems and methods may be used to synthesize various active ingredients for agricultural chemicals, improving their efficacy and stability. Hydrogenation with the above-described systems and methods may be used in the fine chemical industries to produce specific alcohols and other compounds that serve as ingredients in fragrances and flavors. Hydrogenation with the above-described systems and methods may be used to synthesize and/or modify certain dyes and pigments to achieve desired color properties.

Hydrogenation with the above-described systems and methods may also be used in the pharmaceutical industry for the synthesis of various drugs, intermediates, and active pharmaceutical ingredients. For example, nitro groups (—NO₂) can be hydrogenated to amines (—NH₂), which are important building blocks for many drugs. Aldehydes and ketones can be hydrogenated to primary and secondary alcohols, respectively, which are useful intermediates in drug synthesis. Imines (C═N) can be hydrogenated to amines, useful in various pharmaceutical applications. Hydrogenation of aromatic compounds can be implemented to modify the pharmacokinetic properties of drugs, making them more hydrophilic and improving their bioavailability. Hydrogenation can be used for the protection and deprotection of certain functional groups during multi-step pharmaceutical synthesis processes (e.g., to improve the stability of sensitive intermediates).

Hydrogenation in the pharmaceutical industry can provide high selectivity in chemical reactions, which can be useful for producing specific active enantiomers or isomers of drugs, enhancing their efficacy and safety. Hydrogenation can yield high-purity products, which is beneficial for pharmaceutical applications where impurities can lead to adverse effects or reduced efficacy. Further, hydrogenation reactions can be carried out efficiently with the above-describe systems and methods under relatively mild conditions (temperature and pressure), often resulting in higher yields and fewer by-products. Hydrogenation processes are applicable to a wide range of functional groups, making it a versatile tool in the synthesis of diverse pharmaceutical compounds. The processes can be cost-effective compared to other reduction methods, particularly when using reusable catalytic materials and/or catalysts, such as the catalytic tubes described above. For reuse, the tubes may be cleaned and/or recoated with a layer of catalytic material to recycle or reuse the catalyst.

In one example, hydrogenation is used to produce enantiomerically pure compounds used as intermediates in the synthesis of drugs with specific desired activities. Enantiomers are molecules that are mirror images of each other but cannot be superimposed, like left and right hands. Enantiomerically pure drugs often have significantly different biological activities, with one enantiomer being therapeutically active and the other possibly inactive or even harmful. In another example, ibuprofen and other non-steroidal anti-inflammatory drugs (NSAIDs) often involve hydrogenation steps to reduce specific functional groups. Many antidepressants, such as fluoxetine (Prozac®), involve hydrogenation in their synthesis pathways to achieve the desired chemical structure and activity. Hydrogenation is used in producing enantiomerically pure drugs such as certain beta-blockers (e.g., atenolol) and anti-hypertensives (e.g., enalapril). Even synthesis of antibiotics like linezolid and antifungals such as itraconazole involves hydrogenation steps to form useful intermediates.

The above-described array of hydrogenation processes can benefit from the systems and methods of the present application which can provide more controlled hydrogenation reactions.

### 4. Conclusion

While various inventive implementations have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive implementations described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain, using no more than routine experimentation, many equivalents to the specific inventive implementations described herein. It is, therefore, to be understood that the foregoing implementations are presented by way of example only and that inventive implementations may be practiced otherwise than as specifically described. Inventive implementations of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

Also, various inventive concepts may be embodied as one or more methods, of which an example has been provided. The acts performed as part of the method may be ordered in any suitable way. Accordingly, implementations may be constructed in which acts are performed in an order different than illustrated, which may include performing some acts simultaneously, even though shown as sequential acts in illustrative implementations.

All definitions, as defined and used herein, should be understood to control over dictionary definitions, definitions in documents incorporated by reference, and/or ordinary meanings of the defined terms.

Unless stated otherwise, the terms “approximately” and “about” are used to mean within ±20% of a target (e.g., dimension or orientation) in some implementations, within ±10% of a target in some implementations, within ±5% of a target in some implementations, and yet within ±2% of a target in some implementations. The terms “approximately” and “about” can include the target. The term “essentially” is used to mean within ±3% of a target.

The indefinite articles “a” and “an,” as used herein, unless clearly indicated to the contrary, should be understood to mean “at least one.”The phrase “and/or,” as used herein, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of” or “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” shall have its ordinary meaning as used in the field of patent law.

As used herein, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. An apparatus for controlling a hydrogenation reaction, the apparatus comprising:
a reaction chamber to hold molecular hydrogen and at least one compound;
a hydrogenation catalyst, disposed in the reaction chamber, to chemisorb at least the at least one compound and catalyze the hydrogenation reaction, the hydrogenation catalyst comprising a transition metal lattice;
a supply, electrically coupled to the hydrogenation catalyst, to drive an electric current of charged particles through the transition metal lattice that significantly increases vibrations of atoms in the transition metal lattice so as to deliver an energy to the hydrogenation catalyst and thereby facilitate the hydrogenation reaction via propagation of the energy through the transition metal lattice;
at least one sensor to measure at least one of a temperature or a pressure in the reaction chamber associated with the hydrogenation reaction; and
a control system, communicatively coupled to the supply, to control the supply so as to adjust the electric current, based at least in part on the at least one of the temperature or the pressure measured by the at least one sensor, to control the energy delivered to and propagating through the transition metal lattice so as to prevent overheating of the hydrogenation reaction.

2. The apparatus of claim 1, further comprising:
a reaction vessel to contain the reaction chamber;
at least one controllable input port, responsive to the control system, to allow the molecular hydrogen and the at least one compound to flow into the reaction chamber; and
at least one output port, responsive to the control system, to allow at least one reaction product to exit the reaction chamber.

3. The apparatus of claim 1, wherein the supply applies a voltage across the transition metal lattice to drive the electric current.

4. The apparatus of claim 1, wherein the control system controls the supply to:
pulse the electric current, wherein the pulsed electric current has a pulse duration and a pulse frequency; and
the control system controls the supply to adjust at least one of the pulse duration or the pulse frequency of the pulsed electric current to control the energy delivered to the transition metal lattice.

5. The apparatus of claim 4, wherein the transition metal lattice has a cylindrical surface.

6. The apparatus of claim 5, wherein the at least one transition metal lattice includes at least one of palladium, nickel, tungsten, copper, or platinum.

7. The apparatus of claim 6, wherein:
the hydrogenation catalyst further comprises a transmission line, coupled to the supply, to conduct the electric current; and
the transition metal lattice constitutes a portion of a transmission line.

8. The apparatus of claim 1, wherein the control system controls the supply to adjust the electric current to maintain a desired temperature for the hydrogenation reaction.

9. The apparatus of claim 1, wherein the control system controls the supply to induce dissociation of the molecular hydrogen to generate single hydrogen atoms.

10. The apparatus of claim 1, wherein the transition metal lattice includes a self-supporting shape of solid or sintered material.

11. The apparatus of claim 1, wherein the hydrogenation catalyst includes a layer of the transition metal lattice deposited on a substrate.

12. The apparatus of claim 11, wherein the substrate is a ceramic substrate.

13. The apparatus of claim 1, wherein the hydrogenation catalyst has a cylindrical surface.

14. The apparatus of claim 1, wherein the hydrogenation catalyst has a planar surface.

15. The apparatus of claim 1, wherein:
the hydrogenation catalyst further comprises a transmission line, coupled to the supply, to conduct the electric current; and
the transition metal lattice constitutes a portion of a transmission line.

16. The apparatus of claim 1, wherein the at least one transition metal lattice includes at least one of palladium, nickel, tungsten, copper, or platinum.

17. The apparatus of claim 1, wherein the at least one compound includes at least one organic molecule.

18. A method for catalyzing a hydrogenation reaction via a hydrogenation catalyst comprising a transition metal lattice, the method comprising:
A) driving an electric current of charged particles through the transition metal lattice so as to significantly increase vibrations of atoms in the transition metal lattice and thereby propagate an energy through the transition metal lattice to facilitate the hydrogenation reaction between molecular hydrogen and at least one compound chemisorbed by the transition metal lattice;
B) measuring at least one of a temperature or a pressure associated with the hydrogenation reaction; and
C) adjusting the electric current in A) to control the energy propagating through the transition metal lattice, based at least in part on the at least one of the temperature or the pressure measured in B), so as to prevent overheating of the hydrogenation reaction.

19. The method of claim 18, wherein A) comprises applying a voltage across the transition metal lattice.

20. The method of claim 18, wherein C) comprises:
C1) pulsing the electric current, wherein the pulsed electric current has a pulse duration and a pulse frequency; and
C2) adjusting at least one of the pulse duration or the pulse frequency of the pulsed electric current to control the energy delivered to the hydrogenation catalyst.

21. The method of claim 18, wherein C) comprises adjusting the electric current to maintain a desired temperature for the hydrogenation reaction.

22. The method of claim 18, further comprising:
D) inducing dissociation of the molecular hydrogen to generate single hydrogen atoms,
wherein A) comprises facilitating the hydrogenation reaction based at least in part on the energy and the single hydrogen atoms generated in D).

23. The method of claim 22, wherein D) occurs before A).

24. The method of claim 22, wherein D) comprises inducing electrolysis of the molecular hydrogen to generate the single hydrogen atoms.

25. The method of claim 18, wherein in A), the hydrogenation catalyst comprises a layer of the transition metal lattice deposited on a substrate.

26. The method of claim 25, wherein the substrate is a ceramic substrate.

27. The method of claim 18, wherein in A), the hydrogenation catalyst has a cylindrical surface.

28. The method of claim 18, wherein in A), the hydrogenation catalyst has a planar surface.

29. The method of claim 18, wherein:
the hydrogenation catalyst further comprises a transmission line to conduct the electric current; and
the transition metal lattice constitutes a portion of a transmission line.

30. The method of claim 18, wherein the at least one transition metal lattice includes at least one of palladium, nickel, tungsten, copper, or platinum.

31. The method of claim 18, wherein in A), the at least one compound includes at least one organic molecule, and wherein A) comprises:
facilitating the hydrogenation reaction between molecular hydrogen and the at least one organic molecule chemisorbed by the transition metal lattice, based at least in part on the energy delivered in A).

32. The method of claim 31, wherein A) comprises:
driving the electric current through the transition metal lattice so as to facilitate the hydrogenation reaction between molecular hydrogen and the at least one organic molecule to process at least one unsaturated oil or fat.

33. The method of claim 31, wherein in A), the at least one organic molecule includes a nitro compound or an imine, and wherein A) comprises:
driving the electric current through the transition metal lattice so as to facilitate the hydrogenation reaction between molecular hydrogen and the nitro compound or the imine to form an amine.

34. The method of claim 31, wherein in A), the at least one organic molecule includes a carbonyl compound, and wherein A) comprises:
driving the electric current through the transition metal lattice so as to facilitate the hydrogenation reaction between molecular hydrogen and the carbonyl compound to form an alcohol.

35. The method of claim 31, wherein in A), the at least one organic molecule includes an aromatic compound.

36. The method of claim 31, wherein A) comprises:
driving the electric current through the transition metal lattice so as to facilitate the hydrogenation reaction between molecular hydrogen and the at least one organic molecule to synthesize at least one non-steroidal anti-inflammatory drug (NSAID).

37. The method of claim 31, wherein A) comprises:
driving the electric current through the transition metal lattice so as to facilitate the hydrogenation reaction between molecular hydrogen and the at least one organic molecule to synthesize at least one antidepressant drug.

38. The method of claim 31, wherein A) comprises:
driving the electric current through the transition metal lattice so as to facilitate the hydrogenation reaction between molecular hydrogen and the at least one organic molecule to synthesize at least one enantiomerically pure drug.

39. The method of claim 31, wherein A) comprises:
driving the electric current through the transition metal lattice so as to facilitate the hydrogenation reaction between molecular hydrogen and the at least one organic molecule to synthesize at least one antibiotic drug or at least one antifungal drug.

40. The method of claim 31, wherein A) comprises:
driving the electric current through the transition metal lattice so as to facilitate the hydrogenation reaction between molecular hydrogen and the at least one organic molecule to process petroleum.

41. The method of claim 31, wherein A) comprises:
driving the electric current through the transition metal lattice so as to facilitate the hydrogenation reaction between molecular hydrogen and the at least one organic molecule to process coal.

42. The method of claim 31, wherein A) comprises:
driving the electric current through the transition metal lattice so as to facilitate the hydrogenation reaction between molecular hydrogen and the at least one organic molecule to process at least one bio-oil.

43. The method of claim 31, wherein A) comprises:
driving the electric current through the transition metal lattice so as to facilitate the hydrogenation reaction between molecular hydrogen and the at least one organic molecule to process wastewater.

44. The method of claim 31, wherein in A), the at least one organic molecule includes an aldehyde or a ketone.

45. The method of claim 31, wherein in A), the at least one organic molecule includes an alkene or an alkyne.

46. The method of claim 31, wherein in A), the at least one organic molecule includes a nitrile.

47. The method of claim 31, wherein in A), the at least one organic molecule includes styrene.

48. The method of claim 31, wherein in A), the at least one organic molecule includes vinyl chloride.

49. The method of claim 31, wherein in A), the at least one organic molecule includes a polymer.

50. The method of claim 31, wherein in A), the at least one organic molecule includes a rubber.

51. The method of claim 18, wherein in A), the at least one compound includes at least one inorganic molecule, and wherein A) comprises:
driving the electric current through the transition metal lattice so as to facilitate the hydrogenation reaction between molecular hydrogen and the at least one inorganic molecule.

52. The method of claim 51, wherein in A), the at least one inorganic molecule includes a metal oxide, and wherein A) comprises:
driving the electric current through the transition metal lattice so as to facilitate the hydrogenation reaction between molecular hydrogen and the metal oxide to form a metal.

53. The method of claim 51, wherein in A), the at least one inorganic molecule includes carbon dioxide, and wherein A) comprises:
driving the electric current through the transition metal lattice so as to facilitate the hydrogenation reaction between molecular hydrogen and carbon dioxide to form methane or methanol.

54. The method of claim 51, wherein in A), the at least one inorganic molecule includes a nitrogen compound.

55. The method of claim 51, wherein in A), the at least one inorganic molecule includes a metal halide.
